(19) United States
(12) Patent Application Publication
(10) Pub. No.: US 2020/0110095 A1
(43) Pub. Date: Apr. 9, 2020

(54) METHODS OF PROTEIN INTERACTION ANALYSIS

(71) Applicant: Momenta Pharmaceuticals, Inc., Cambridge, MA (US)

(72) Inventors: James Madsen, Medford, MA (US); Stephen Smith, Cambridge, MA (US)

(21) Appl. No.: 16/546,742

(22) Filed: Aug. 21, 2019

Related U.S. Application Data
(60) Provisional application No. 62/720,502, filed on Aug. 21, 2018.

(51) Int. Cl. G01N 33/68; G01N 33/532

(52) U.S. Cl. CPC G01N 33/6848; G01N 2458/15; G01N 33/532; G01N 33/6842

(57) ABSTRACT

Characterization of proteins and/or protein complexes using covalent labeling denaturation methodology are described.

### CROSS REFERENCE TO RELATED APPLICATIONS

This application claims the benefit of U.S. Provisional Application No. 62/720,502, filed Aug. 21, 2018, which is hereby incorporated by reference in its entirety.

### SEQUENCE LISTING

The instant application contains a Sequence Listing which has been filed electronically in ASCII format and is hereby incorporated by reference in its entirety. Said ASCII copy, created on Dec. 12, 2019, is named 2010403-0532_SL.txt and is 14,760 bytes in size.

### BACKGROUND

Therapeutic polypeptides, including therapeutic antibodies, are an important class of therapeutic biotechnology products. Protein structure and conformational characteristics of a therapeutic protein are important for therapeutic activity.

### SUMMARY OF THE INVENTION

The present disclosure provides, in part, methods for evaluating, identifying, analyzing and/or producing (e.g., manufacturing) a protein, e.g., a glycoprotein, e.g., an antibody, e.g., a fusion protein and/or a protein complex, e.g., a glycoprotein complex, e.g., an antibody-antigen complex, e.g., a fusion-protein complex. In some instances, methods herein allow highly resolved evaluation of protein-protein interactions (e.g., antibody-antigen interactions) useful for, inter alia, identifying binding partners with desired binding characteristics (e.g., binding at a particular site and/or with a particular binding strength), assessing new drugs, and/or manufacturing (e.g., release testing).

The present disclosure encompasses, in part, a recognition that methods utilizing high amounts of label to purposely denature protein complexes can result in decreased labeling protection at protein-protein binding interfaces. The present disclosure provides, at least in part, methods that include exposing one or more proteins and/or protein complexes to a relatively high amount of label and obtaining an MS signal of the labeled proteins and/or protein complexes. The present disclosure provides the insight that such methods can be used to assess local binding sites and/or provide a measure of interaction strength.

In certain aspects, the disclosure provides methods of characterizing protein-protein interactions between two or more proteins in a protein complex. In some aspects, a method of determining a site of protein-protein interaction is provided, where the method comprises: (i) exposing a first sample of a protein-protein complex with a first level of a label to obtain a labeled protein-protein complex in a first state; (ii) exposing a second sample of the protein-protein complex with a second level of the label to obtain a labeled protein-protein complex in a second state, wherein the second level of the label is sufficient to induce a conformational change of the protein-protein complex; (iii) using mass spectrometry to obtain a MS signal of the labeled protein-protein complex in the first state and a MS signal of the labeled protein-protein complex in the second state; and (iv) determining a site of interaction by comparing the MS signals of the labeled protein-protein complex in the first state and the second state.

In some embodiments, a label is a covalent label. In some embodiments, a label is an isobaric label. In some embodiments, an isobaric label is a TMT label.

In some embodiments, a second level of label (i.e., a level of label sufficient to induce a conformational change of the protein-protein complex) is within a range of 100-100,000 molar excess relative to the protein-protein complex.

In some embodiments, using mass spectrometry comprises digesting (e.g., enzymatically) the labeled protein-protein complex to produce a plurality of peptides, which plurality of peptides comprises both labeled and unlabeled peptides. In some embodiments, the plurality of peptides are analyzed by MS, such as, e.g., LC-MS/MS. In some embodiments, using mass spectrometry to obtain a MS signal comprises denaturing, reducing, alkylating, enzymatically digesting, and analyzing by LC-MS/MS. In some embodiments, peptides can be identified by database searching MS/MS spectra, and reporter ion ratios are used to calculate fold changes (i.e., localized structural deviations) for each peptide.

In some embodiments, a site of interaction corresponds to a sequence of the first protein and/or second protein that is protected from labeling (e.g., protected from labeling in the first state, but not in the second state). In some embodiments, a sequence that is protected from labeling is a sequence of unlabeled peptide(s) obtained by digesting the labeled complex, where peptides are unlabeled in the first state and the corresponding peptides are labeled in the second state.

In some embodiments, a protein-protein complex comprises a first protein and a second protein. In some embodiments, a method of determining a site of protein-protein interaction further comprises exposing a sample of first protein and/or a sample of second protein to a label to determine the sequences of the exposed polypeptide surfaces of the individual proteins. In some embodiments, a site of interaction corresponds to a sequence of first protein and/or second protein that is protected from labeling when in the protein-protein complex, but is accessible to labeling as a free protein. In some embodiments, a sequence that is protected from labeling is sequence of unlabeled peptide(s) obtained by digesting the labeled complex, where peptides are unlabeled in the complex and the corresponding peptides from free protein are labeled.

In some embodiments, a first and/or second protein is glycosylated.

In some embodiments, provided methods further include: exposing the protein-protein complex in the first state to a third level of label to obtain a labeled protein-protein complex in a third state, where the third level is sufficient to induce a conformational change of the protein-protein complex; using mass spectrometry to obtain a MS signal of the labeled protein-protein complex in the third state; comparing the MS signal of the labeled protein-protein complex in the first, second, and third states to assess binding strength of the first protein to the second protein at one or more sites of interaction.

In some aspects, a method of characterizing protein-protein interactions is provided, where the method comprises: (i) providing a sample of a protein-protein complex comprising a first protein and a second protein; (ii) exposing the protein-protein complex to 2 or more levels of label to obtain labeled protein-protein complexes in 2 or more states, wherein each state corresponds to a level of label, and wherein at least one level of label induces a conformational change of the protein-protein complex; (iii) using mass spectrometry to obtain a MS signal for each of the 2 or more states of labeled protein-protein complex; and (iv) comparing the MS signals to characterize one or more sites of interaction between the first and second protein of the protein complex.

In some embodiments, a label is a covalent label. In some embodiments, a label is an isobaric label. In some embodiments, an isobaric label is a TMT label.

In some embodiments, second level of the label (i.e., a level of label sufficient to induce a conformational change of the protein-protein complex) is within a range of 100-100,000 molar excess relative to the protein-protein complex.

In some embodiments, a characterizing one or more sites of interaction between the first and second protein of the protein complex comprises determining an amino acid sequence of a site of interaction. In some embodiments, a site of interaction comprises a sequence of the first protein and/or second protein that is protected from labeling in one or more states.

In some embodiments, provided methods further comprise determining a strength of interaction between the first protein and the second protein at one or more sites of interaction.

In some embodiments, a protein-protein complex is exposed to 3, 4, 5, 6, 7, 8, 9, 10 or more levels of label.

In some embodiments, a method of characterizing protein-protein interactions comprises: (i) exposing the protein-protein complex to 3 or more levels of label to obtain labeled protein-protein complexes in 3 or more states, wherein each state corresponds to a level of label, and wherein at least 2 levels of label induce a conformational change of the protein-protein complex; (ii) using mass spectrometry to obtain a MS signal for each of the 3 or more states of labeled protein-protein complex; (iii) comparing the MS signals for each of the 3 or more different states to determine a strength of interaction between the first protein and the second protein at one or more sites of interaction. In some embodiments, a strength of interaction correlates with an amount of label needed to disrupt the interaction and/or expose the surface for labeling.

In some embodiments, using mass spectrometry comprises digesting (e.g., enzymatically) the labeled protein-protein complex to produce a plurality of peptides, which plurality of peptides comprises both labeled and unlabeled peptides. In some embodiments, the plurality of peptides are analyzed by MS, such as, e.g., LC-MS/MS. In some embodiments, using mass spectrometry to obtain a MS signal comprises denaturing, reducing, alkylating, enzymatically digesting, and analyzing by LC-MS/MS. In some embodiments, peptides can be identified by database searching MS/MS spectra, and reporter ion ratios are used to calculate fold changes (i.e., localized structural deviations) for each peptide.

In some embodiments, a site of interaction corresponds to a sequence of the first protein and/or second protein that is protected from labeling (e.g., protected from labeling in one state, but exposed to label in another state (e.g., a state exposed to high amount of label)). In some embodiments, a sequence that is protected from labeling is a sequence of unlabeled peptide(s) obtained by digesting the labeled complex, where peptides are unlabeled in one state and the corresponding peptides are labeled in other state(s).

In some embodiments, a protein-protein complex comprises a first protein and a second protein. In some embodiments, a method of determining a site of protein-protein interaction further comprises exposing a sample of first protein and/or a sample of second protein to a label to determine the sequences of the exposed polypeptide surfaces of the individual proteins. In some embodiments, a site of interaction corresponds to a sequence of first protein and/or second protein that is protected from labeling when in the protein-protein complex, but is accessible to labeling as a free protein. In some embodiments, a sequence that is protected from labeling is sequence of unlabeled peptide(s) obtained by digesting the labeled complex, where peptides are unlabeled in the complex and the corresponding peptides from free protein are labeled.

In some embodiments, a first and/or second protein is glycosylated.

In some aspects, a method of identifying and/or screening a protein binding partner is provided, where the method comprises: (i) providing a sample of a protein; (ii) contacting the sample of the protein with a test protein to form a protein-test protein complex; (iii) exposing the protein-test protein complex to 2 or more levels of label to obtain labeled protein-protein complexes in 2 or more states, where each state corresponds to a level of label, and wherein at least one level of label induces a conformational change of the protein-test protein complex; (iv) using mass spectrometry to obtain a MS signal for each of the 2 or more states of labeled protein-test protein complex; (v) determining (1) a site of interaction by comparing the MS signals of the 2 or more states of labeled protein-test protein complex and/or (2) a strength of interaction between the protein and the test protein at one or more sites of interaction; and (vi) selecting the test protein as a protein binding partner if the site of interaction and/or strength of interaction is tolerable.

In some embodiments, a site of interaction is a sequence of the protein that is protected from labeling.

In some embodiments, a site of interaction is tolerable when it overlaps a desired or predetermined site of interaction between the protein and the protein binding partner. In some embodiments, a site of interaction is tolerable when the sequence of the protein binding partner that is protected from labeling is 80%, 85%, 90% 95%, 98%, 99% or 100% identical to a desired or predetermined sequence of interaction.

In some embodiments, a label is a covalent label. In some embodiments, a label is an isobaric label. In some embodiments, an isobaric label is a TMT label.

In some embodiments, second level of the label (i.e., a level of label sufficient to induce a conformational change of the protein-test protein complex) is within a range of 100-100,000 molar excess relative to the protein-test protein complex.

In some embodiments, using mass spectrometry comprises digesting (e.g., enzymatically) the labeled protein-protein complex to produce a plurality of peptides, which plurality of peptides comprises both labeled and unlabeled peptides. In some embodiments, the plurality of peptides are analyzed by MS, such as, e.g., LC-MS/MS. In some embodiments, using mass spectrometry to obtain a MS signal comprises denaturing, reducing, alkylating, enzymatically digesting, and analyzing by LC-MS/MS. In some embodiments, peptides can be identified by database searching MS/MS spectra, and reporter ion ratios are used to calculate fold changes (i.e., localized structural deviations) for each peptide.

In some embodiments, a protein and/or test protein are glycosylated.

In some aspects, a method of identifying and/or screening a protein binding partner is provided, where the method comprises: (i) providing a sample of a protein; (ii) contacting the sample of the protein with a test protein to form a protein-test protein complex; (iii) exposing a first sample of a protein-test protein complex with a first level of a label to obtain a labeled protein-test protein complex in a first state; (iii) exposing a second sample of the protein-test protein complex with a second level of the label to obtain a labeled protein-test protein complex in a second state, wherein the second level of the label is sufficient to induce a conformational change of the protein-test protein complex; (iv) using mass spectrometry to obtain a MS signal of the labeled protein-test protein complex in the first state and a MS signal of the labeled protein-test protein complex in the second state; (v) determining a site of interaction by comparing the MS signals of the labeled protein-test protein complex in the first state and the second state; and (vi) selecting the test protein as a protein binding partner if the site of interaction is tolerable.

In some embodiments, a site of interaction is a sequence of the protein that is protected from labeling.

In some embodiments, a site of interaction is tolerable when it overlaps a desired or predetermined site of interaction between the protein and the protein binding partner. In some embodiments, a site of interaction is tolerable when the sequence of the protein binding partner that is protected from labeling is 80%, 85%, 90% 95%, 98%, 99% or 100% identical to a desired or predetermined sequence of interaction.

In some embodiments, a label is a covalent label. In some embodiments, a label is an isobaric label. In some embodiments, an isobaric label is a TMT label.

In some embodiments, second level of the label (i.e., a level of label sufficient to induce a conformational change of the protein-test protein complex) is within a range of 100-100,000 molar excess relative to the protein-test protein complex.

In some embodiments, using mass spectrometry comprises digesting (e.g., enzymatically) the labeled protein-protein complex to produce a plurality of peptides, which plurality of peptides comprises both labeled and unlabeled peptides. In some embodiments, the plurality of peptides are analyzed by MS, such as, e.g., LC-MS/MS. In some embodiments, using mass spectrometry to obtain a MS signal comprises denaturing, reducing, alkylating, enzymatically digesting, and analyzing by LC-MS/MS. In some embodiments, peptides can be identified by database searching MS/MS spectra, and reporter ion ratios are used to calculate fold changes (i.e., localized structural deviations) for each peptide.

In some embodiments, a protein and/or test protein are glycosylated.

The present disclosure encompasses the recognition that provided methods may be useful for release testing and/or validation of protein products in a method of manufacture. In certain aspects, the disclosure provides methods of manufacturing. Such methods can include providing (e.g., producing, expressing (e.g., in small scale or large scale cell culture) and/or manufacturing) or obtaining (e.g., receiving and/or purchasing from a third party (including a contractually related third party or a non-contractually-related (e.g., an independent) third party)) a test protein (e.g., a test protein drug substance, e.g., a batch of a test protein drug substance).

In some aspects, a method of manufacture is provided, where the method comprises: (i) providing (e.g., producing, expressing (e.g., in small scale or large scale cell culture) and/or manufacturing) or obtaining (e.g., receiving and/or purchasing from a third party (including a contractually related third party or a non-contractually-related (e.g., an independent) third party)) a test protein drug substance (e.g., a sample of a test protein or a batch of test protein), (ii) exposing a sample of the test protein with a protein binding partner to form a test protein-protein complex in a first state, (iii) exposing the test protein-protein complex in a first state to a stressor to obtain a labeled test protein-protein complex in a second state, (iv) using mass spectrometry to obtain a test MS signal of the labeled test protein-protein complex in the first state and the second state, (v) determining a site of interaction by comparing the test MS signal of the labeled test protein-protein complex in the first state and the second state, and (vi) (a) processing the test protein drug substance as drug product (e.g., processing the batch of test protein drug substance) if the site of interaction is tolerable, or (b) taking an alternative action if the site of interaction is not tolerable.

In some embodiments, in some embodiments, a step of using mass spectrometry also includes digesting a labeled test protein-protein complex to produce a plurality of labeled test peptides.

In some embodiments, a stressor is a label. In some embodiments, a label is an isobaric label. In some certain embodiments, an isobaric label is a TMT label.

In some embodiments, a stressor is a label that is provided at a concentration that is sufficient to induce a conformational change of a test protein-protein complex. In some embodiments, a concentration of label is sufficient to induce a conformational change of a test protein in a test protein-protein complex. In some embodiments, a concentration of label is sufficient to induce a conformational change of a protein binding partner in a test protein-protein complex. In some embodiments, a concentration of label is sufficient to induce a conformational change of both a test protein and a protein binding partner in a test protein-protein complex.

In some embodiments, a stressor is a label that is provided within a range of 100 to 100,000 molar excess relative to a test protein-protein complex. In some embodiments, a stressor is a label that is provided within a range of 100 to 10,000 molar excess relative to a test protein-protein complex. In some embodiments, a stressor is a label that is provided within a range of 500 to 5,000 molar excess relative to a test protein-protein complex. In some certain embodiments, a stressor is a label that is provided within a range of 500 to 1,000 molar excess relative to a test protein-protein complex.

In some embodiments, a site of interaction for a test protein-protein complex is considered to be tolerable when it overlaps a known and/or determined site of interaction between a target protein and a protein binding partner (e.g., the same protein binding partner that is included in a test protein-protein complex). In some embodiments, a target protein is approved under a primary approval process. In some embodiments, a target protein has an amino acid sequence that is at least 95%, 96%, 97%, 98%, or 99% identical to a test protein. In some embodiments, a target protein has an amino acid sequence that is 100% identical to a test protein. In some embodiments, a target protein is approved under a BLA and has an amino acid sequence that is 100% identical to a test protein.

In some embodiments, a site of interaction is a sequence of a protein binding partner that is protected from labeling. In some embodiments, a site of interaction is tolerable when the sequence of a protein binding partner that is protected from labeling is 80%, 85%, 90% 95%, 98%, 99% or 100% identical to a known and/or determined sequence of interaction between a target protein and the protein binding partner. In some embodiments, a site of interaction is tolerable when the sequence of a protein binding partner that is protected from labeling is 80% to 100% identical, 90% to 100% identical, or 95% to 100% identical to a known and/or determined sequence of interaction between a target protein and the protein binding partner.

In some embodiments, a site of interaction is a sequence of a test protein that is protected from labeling. In some embodiments, a site of interaction is tolerable when the sequence of a test protein that is protected from labeling is 80%, 85%, 90% 95%, 98%, 99% or 100% identical to a known and/or determined sequence of interaction between a target protein and an identical protein binding partner. In some embodiments, a site of interaction is tolerable when the sequence of a test protein that is protected from labeling is 80% to 100% identical, 90% to 100% identical, or 95% to 100% identical to a known and/or determined sequence of interaction between a target protein and the protein binding partner.

In some embodiments, a site of interaction is not tolerable when it does not overlap with a known or determined site of interaction between a target protein and its protein binding partner. In certain some embodiments, a site of interaction is not tolerable when the sequence of a test protein and/or a protein binding partner that is protected from labeling is less than 80%, 75%, 70%, 65%, 60%, 55%, 50%, 45%, 40%, 35%, 30%, or 25% identical to a known and/or determined sequence of interaction between a target protein and an identical protein binding partner.

In some embodiments, a site of interaction is considered not tolerable and an alternative action includes one or more of: disposing of a test protein, classifying a test protein for disposal, labeling a test protein for disposal, and reprocessing a test protein.

In some embodiments, a test protein is or comprises an Fc fusion protein or antibody. In some embodiments, a test protein is glycosylated.

In some embodiments, a target protein is or comprises an Fc fusion protein or an antibody. In some embodiments, a target protein is glycosylated.

In some embodiments, a protein binding partner is a protein ligand, receptor, antigen, and/or an enzyme. In some embodiments, a protein binding partner is glycosylated.

In some embodiments, a site of interaction is considered tolerable and a processing step comprises one or more of: formulating a test protein; combining a test protein with a second component, e.g., an excipient or buffer; changing the concentration of a test protein in a preparation; lyophilizing a test protein; combining a first and second aliquot of a test protein to provide a third, larger, aliquot; dividing a test protein into smaller aliquots; disposing a test protein into a container, e.g., a gas or liquid tight container; packaging a test protein; associating a container comprising a test protein with a label (e.g., labeling); shipping or moving a test protein to a different location.

In some embodiments, a method of manufacture also includes: (i) exposing a second sample of a test protein with the protein binding partner to form a second test protein-protein complex, (ii) exposing the second test protein-protein complex to label at a second concentration, (iii) using mass spectrometry to obtain a second test MS signal of the labeled second test protein-protein complex, (iv) comparing the first test MS signal to the second test MS signal to assess binding strength of the test protein to the protein binding partner at a particular site on the protein binding partner, and (v) (a) processing the batch of the test protein drug substance as drug product if the binding strength is tolerable, or (b) taking an alternative action if the binding strength is not tolerable.

In some embodiments, the binding strength is considered tolerable when it meets a predetermined value. In some embodiments, the binding strength is considered tolerable when it differs by no more than 30%, 20% or 10% from a known and/or determined binding strength of a target protein to the protein binding partner at the particular site.

In some embodiments, the binding strength is considered not tolerable and an alternative action includes one or more of: disposing of a first and/or second test protein, classifying a first and/or second test protein for disposal, labeling a first and/or second test protein for disposal, and reprocessing a first and/or second test protein.

In some embodiments, a first and/or second test protein is or comprises an Fc fusion protein or antibody. In some embodiments, a first and/or second test protein is glycosylated.

In some embodiments, a protein binding partner is a protein ligand, receptor, antigen, and/or an enzyme. In some embodiments, a protein binding partner is glycosylated.

In some embodiments, the binding strength is considered tolerable and a processing step comprises one or more of: formulating a test protein; combining a test protein with a second component, e.g., an excipient or buffer; changing the concentration of a test protein in a preparation; lyophilizing a test protein; combining a first and second aliquot of a test protein to provide a third, larger, aliquot; dividing a test protein into smaller aliquots; disposing a test protein into a container, e.g., a gas or liquid tight container; packaging a test protein; associating a container comprising a test protein with a label (e.g., labeling); shipping or moving a test protein to a different location.

In some aspects, a method of manufacture is provided that comprises: (i) providing (e.g., producing, expressing (e.g., in small scale or large scale cell culture) and/or manufacturing) or obtaining (e.g., receiving and/or purchasing from a third party (including a contractually related third party or a non-contractually-related (e.g., an independent) third party)) a test protein drug substance (e.g., a sample of a test protein or a batch of test protein); (ii) determining or obtaining a determination of a contact site between a sample of the test protein and a protein binding partner; and (iii) (a) processing the test protein drug substance (e.g., processing a corresponding batch of test protein drug substance) as drug product if the contact site sufficiently matches that for a target protein and the protein binding partner; or (b) taking an alternative action if the contact site does not sufficiently match that for a target protein and the protein binding partner, where the contact site between a sample of a test protein and a protein binding partner is determined by: (iv) exposing a sample of the test protein with a protein binding partner to form a test protein-protein complex; (v) exposing the test protein-protein complex to label at a concentration sufficient to induce a conformational change; and (vi) using mass spectrometry to obtain a test MS signal of the labeled test protein-protein complex.

In some embodiments, a determination of a contact site between a sample of a test protein and a protein binding partner also includes comparing the test MS signal to a target MS signal for a target protein drug product complexed with the same protein binding partner (i.e. a protein with an identical amino acid sequence as the protein binding partner) and exposed to label at the same concentration.

In some embodiments, a concentration of label is sufficient to induce a conformational change of a test protein in a test protein-protein complex. In some embodiments, a concentration of label is sufficient to induce a conformational change of a protein binding partner in a test protein-protein complex. In some embodiments, a concentration of label is sufficient to induce a conformational change of both a test protein and a protein binding partner in a test protein-protein complex.

In some embodiments, a step of using mass spectrometry to obtain a test MS signal of the labeled test protein-protein complex also includes digesting the labeled test protein-protein complex to produce a plurality of labeled test peptides.

In some embodiments, a method of manufacture also includes producing a representation of a comparison of the test MS signal and the target MS signal.

In some embodiments, a label is an isobaric label. In some embodiments, an isobaric label is a TMT label.

In some embodiments, a target protein is approved under a BLA. In some embodiments, a target protein has an amino acid sequence that is at least 95%, 96%, 97%, 98%, or 99% identical to a test protein. In some embodiments, a target protein has an amino acid sequence that is 100% identical to a test protein. In some embodiments, a target protein is approved under a BLA and has an amino acid sequence that is 100% identical to a test protein.

In some embodiments, a contact site is an amino acid sequence of a protein binding partner and/or a test protein that is protected from labeling. In some embodiments, a contact site for a test protein sufficiently matches that of a target protein if the contact site matches 90%, 95%, 98%, 99% or 100% of amino acid residues of the sequence of protein binding partner bound by the target protein. In some embodiments, a contact site for a test protein sufficiently matches that of a target protein if the contact site matches 90%, 95%, 98%, 99% or 100% of amino acid residues of the sequence of test protein bound by the protein binding partner.

In some embodiments, a contact site for a test protein sufficiently matches that of a target protein and a processing step comprises one or more of: formulating a test protein; combining a test protein with a second component, e.g., an excipient or buffer; changing the concentration of a test protein in a preparation; lyophilizing a test protein; combining a first and second aliquot of a test protein to provide a third, larger, aliquot; dividing a test protein into smaller aliquots; disposing a test protein into a container, e.g., a gas or liquid tight container; packaging a test protein; associating a container comprising a test protein with a label (e.g., labeling); shipping or moving a test protein to a different location.

In some embodiments, a contact site does not sufficiently match that of a known or determined contact site between a target protein and its protein binding partner when the sequence of a test protein and/or a protein binding partner that is protected from labeling is less than 80%, 75%, 70%, 65%, 60%, 55%, 50%, 45%, 40%, 35%, 30%, or 25% identical to a known and/or determined contact site between a target protein and the protein binding partner.

In some embodiments, a contact site is considered not sufficient and an alternative action includes one or more of: disposing of the test protein, classifying for disposal the test protein, labeling the test protein for disposal, and reprocessing the test protein.

In some embodiments, a concentration of label sufficient to induce a conformational change is within a range of 100 to 100,000 molar excess. In some embodiments, a concentration of label sufficient to induce a conformational change is within a range of 100 to 10,000 molar excess. In some embodiments, a concentration of label sufficient to induce a conformational change is within a range of 500 to 5,000 molar excess. In some certain embodiments, a concentration of label sufficient to induce a conformational change is within a range of 500 to 1,000 molar excess.

In some embodiments, a test protein is or comprises an Fc fusion protein or antibody. In some embodiments, a test protein is glycosylated.

In some embodiments, a target protein is or comprises an Fc fusion protein or an antibody. In some embodiments, a target protein is glycosylated.

In some embodiments, a protein binding partner is a protein ligand, receptor, antigen, and/or an enzyme. In some embodiments, a protein binding partner is glycosylated.

In some embodiments, a method of manufacture also includes: (i) determining or obtaining a determination of the strength of interaction between a sample of a test protein and a protein binding partner, where the determination of the strength of interaction comprises: (a) exposing a second sample of the test protein with the protein binding partner to form a second test protein-protein complex; (b) exposing the second test protein-protein complex to label at a second concentration; (c) using mass spectrometry to obtain a second test MS signal of the labeled second test protein-protein complex; (d) comparing the first test MS signal to the second test MS signal to assess binding strength of the test protein to the protein binding partner; and (ii) processing the batch of the test protein drug substance as drug product if the binding strength is tolerable; or taking an alternative action if the binding strength is not tolerable.

In some embodiments, a binding strength is considered tolerable when it meets a predetermined value. In some embodiments, the binding strength is considered tolerable when it differs by no more than 30%, 20% or 10% from a known and/or determined binding strength of a target protein to the protein binding partner at the particular site.

In some embodiments, a binding strength is considered tolerable and a processing step comprises one or more of: formulating a test protein; combining a test protein with a second component, e.g., an excipient or buffer; changing the concentration of a test protein in a preparation; lyophilizing a test protein; combining a first and second aliquot of a test protein to provide a third, larger, aliquot; dividing a test protein into smaller aliquots; disposing a test protein into a container, e.g., a gas or liquid tight container; packaging a test protein; associating a container comprising a test protein with a label (e.g., labeling); shipping or moving a test protein to a different location.

In some embodiments, a binding strength is considered not tolerable and an alternative action includes one or more of: disposing of a first and/or second test protein, classifying a first and/or second test protein for disposal, labeling a first and/or second test protein for disposal, and reprocessing a first and/or second test protein.

In any of the aspects described herein, methods can further include, e.g., one or more of: memorializing a comparison and/or results of a comparison (e.g., between one or more test MS signal(s) and one or more target MS signal(s)) using a recordable medium (e.g., on paper or in a computer readable medium, e.g., in a Certificate of Testing, Material Safety Data Sheet (MSDS), batch record, or Certificate of Analysis (CofA)); informing a party or entity (e.g., a contractual or manufacturing partner, a care giver or other end-user, a regulatory entity, e.g., the FDA or other U.S., European, Japanese, Chinese or other governmental agency, or another entity, e.g., a compendial entity (e.g., U.S. Pharmacopoeia (USP)) or insurance company) of the comparison and/or results of the comparison.

These, and other aspects of the invention, are described in more detail below and in the claims.

### BRIEF DESCRIPTION OF THE DRAWING

The Drawing included herein, which is composed of the following Figures, is for illustration purposes only and not for limitation.

FIG. 1 panels (A) and (B) describe changes in relative abundance of labeled peptides from model antibody-antigen complexes exposed to increasing concentrations of isobaric label. FIG. 1 panel (C) describes localized protection of regions particular model antigen regions with increasing concentrations of isobaric label. For FIG. 1 panels (A), (B), and (C), unique TMT labeled peptides are arranged from lowest to highest fold change. A fold change near one indicates equivalence between the two samples for a given TMT labeled peptide.

FIG. 2 depicts dose-response curves for TMT-labeled peptides for an antigen of a model antibody-antigen complex after reaction with increasing amounts of TMT labeling agent.

FIG. 3 depicts localized covalent labeling denaturation structural assessment for panel (A) model antigen (TNFα) alone versus model antibody-antigen complex (TNFα/IgG1(a)) and panel (B) model antigen (TNFα) alone versus antigen with a nonspecific antibody.

FIG. 4 depicts TMT-labeled peptide sequence coverage for model antigen (TNFα, top) and structural assessment (bottom, PDB: 3WD5) from covalent labeling denaturation of model antigen alone versus model antibody-antigen complex. Blue highlights indicate the areas where antigen was more protected from the label in the antibody-antigen complex (negative fold changes), red highlights indicate that both negative and positive fold changes were observed for the same region, and purple highlights specify negligible fold changes between the samples (i.e., label protection was similar for antigen alone versus antibody-antigen complex). Yellow letters represent the epitope sites previously reported using crystallography. FIG. 4 discloses SEQ ID NO: 47.

FIG. 5 depicts in panel (A) TMT-labeled peptide sequence coverage for a model antigen (TNFα, top) and localized structural assessment (bottom) for model antibody-antigen complex with a first antibody versus with a second antibody under denaturing labeling conditions. In FIG. 5 panel (B) TMT-labeled peptide sequence coverage for antigen (top) and localized structural assessment (bottom) for antibody-antigen complexes with the first and second antibodies under nondenaturing labeling conditions. The TMT labeling amounts in (A) and (B) were 5.3 mM and 0.5 mM TMT, respectively. Blue highlights in the sequence coverage maps indicate that antigen was more protected with the first antibody, red highlights indicate that antigen was more protected with the second antibody, and purple highlights specify negligible fold changes between the samples. Yellow letters represent the epitope sites previously reported using crystallography. FIG. 5 panels (A) and (B) disclose SEQ ID NO: 47.

FIG. 6 depicts TMT-labeled peptide sequence coverage for a model antigen (top) and structural assessment (bottom, PDB: 3WD5) from covalent labeling denaturation of antibody-antigen complex with a first antibody versus antibody-antigen complex with a second antibody. Blue highlights indicate that model antigen was more protected with the first antibody, red highlights indicate that model antigen was more protected with the second antibody, and purple highlights specify negligible fold changes between the samples. Yellow letters represent the epitope sites previously reported using crystallography. FIG. 6 discloses SEQ ID NO: 47.

FIG. 7 depicts TMT-labeled peptide sequence coverage for a model ligand (B7-1, top) and localized covalent labeling denaturation structural assessment (bottom) for model ligand/Fc-Fusion complexes with a first and second model Fc-Fusion protein (B7-1/Fc-Fusion(a) versus B7-1/Fc-Fusion(b)). Red highlights in the sequence coverage map (top) indicate amino acids of model ligand B7-1 that were more protected with the second Fc-Fusion and purple highlights specify negligible fold changes between the samples. Yellow letters represent the protein-ligand binding sites previously reported using crystallography. FIG. 7 discloses SEQ ID NO: 48.

FIG. 8 depicts TMT-labeled peptide sequence coverage for model ligand (B7-1, top) and structural assessment (bottom, PDB: 118L) from covalent labeling denaturation of model ligand/Fc-Fusion complexes with a first and second model Fc-Fusion protein (B7-1/Fc-Fusion(a) versus B7-1/Fc-Fusion(b)). Red highlights in the sequence coverage map indicate that antigen was more protected with Fc-Fusion(b) and purple highlights specify negligible fold changes between the samples. Yellow letters represent the protein-ligand binding sites previously reported using crystallography. FIG. 8 discloses SEQ ID NO: 48.

FIG. 9 depicts localized covalent labeling denaturation structural assessment for panel (A) model ligand/Fc-Fusion complexes compared with an acidified ligand/Fc-Fusion complex panel (B) model ligand-Fc-Fusion complexes compared with an oxidized ligand/Fc-Fusion complex, and panel (C) model ligand-Fc-Fusion complexes compared with a heated ligand/Fc-Fusion complex.

FIG. 10 depicts TMT-labeled peptide sequence coverage of a model ligand B7-1 for (A) model ligand/Fc-Fusion complexes compared with an acidified ligand/Fc-Fusion complex (B) model ligand-Fc-Fusion complexes compared with an oxidized ligand/Fc-Fusion complex, and (C) model ligand-Fc-Fusion complexes compared with a heated ligand/Fc-Fusion complex. Red highlights indicate that ligand was more protected with stressed Fc-Fusion(a), and purple highlights specify negligible fold changes between the samples. Yellow letters represent the protein-ligand binding sites previously reported using crystallography. FIG. 10 at (A), (B) and (C) all disclose SEQ ID NO: 48.

### CERTAIN DEFINITIONS

As used herein, a “glycoprotein” refers to an amino acid sequence that includes one or more oligosaccharide chains (e.g., glycans) covalently attached thereto. Exemplary amino acid sequences include polypeptides and proteins. Exemplary glycoproteins include glycosylated antibodies, antibody agents, and antibody-like molecules (e.g., Fc fusion proteins). Exemplary antibodies include monoclonal antibodies and/or fragments thereof, polyclonal antibodies and/or fragments thereof, and Fc domain containing fusion proteins (e.g., fusion proteins containing the Fc region of IgG1, or a glycosylated portion thereof).

As used herein, a “batch” in reference to protein preparation refers to a single manufacturing run of the protein. Evaluation of different batches thus means evaluation of different manufacturing runs or batches.

As used herein, “sample(s)” typically refers to an aliquot of material separately obtained, procured or derived from a source of interest. In some embodiments, sample is a protein of interest or preparation thereof. In some embodiments, evaluation of separate samples includes evaluation of different commercially available containers or vials of the same batch or from different batches.

As used herein, “obtain” or “obtaining” (e.g., “obtaining information”) means acquiring possession of a physical entity, a value, e.g., a numerical value, or information, e.g., data, by “directly obtaining” or “indirectly obtaining” the physical entity. value, or information. “Directly obtaining” means performing a process (e.g., performing an assay or test on a sample) to acquire the physical entity or value. “Indirectly obtaining” refers to receiving the physical entity or value from another party or source (e.g., a third party laboratory that directly acquired the physical entity or value). “Directly obtaining” a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. “Directly obtaining” a value and/or information includes performing a process that includes a physical change in a sample or another substance, e.g., performing an analytical process (e.g., an MS process) which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, the term “approximately” or “about,” as applied to one or more values of interest, refers to a value that is similar to a stated reference value. In certain embodiments, the terms “approximately” or “about” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the stated reference value.

In general, a “protein”, as used herein, is a polypeptide (i.e., a string of at least ten amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “protein preparation” as used herein refers to a mixture of proteins obtained according to a particular production method. Proteins in a protein preparation may be the same or different, i.e., a protein preparation may include several copies of the same protein and/or a mixture of different proteins. In some embodiments, a protein preparation includes glycoprotein preparations. A glycoprotein preparation is a composition or mixture that includes at least one glycoprotein. In some instances, a glycoprotein preparation (e.g., such as a glycoprotein drug substance or a precursor thereof) can be a sample from a proposed or test batch of a drug substance or drug product. Production methods generally include a recombinant preparation step using cultured cells that have been engineered to express the proteins in the protein preparation (or to express the proteins at a relevant level or under relevant conditions). A production method may further include an isolation step in which proteins are isolated from certain components of the engineered cells (e.g., by lysing the cells and pelleting the protein component by centrifugation). A production method may also include a purification step in which the proteins in the protein preparation are separated (e.g., by chromatography) from other cellular components, e.g., other proteins or organic components that were used in earlier steps. It will be appreciated that these steps are non-limiting and that any number of additional productions steps may be included. Different protein preparations may be prepared by the same production method but on different occasions (e.g., different batches). Alternatively, different protein preparations may be prepared by different production methods. Two production methods may differ in any way (e.g., expression vector, engineered cell type, culture conditions, isolation procedure, purification conditions, etc.).

As used herein, the terms “biologic”, “biotherapeutic”, and “biologic product” are used interchangeably to refer to polypeptide and protein products. For example, biologics herein include naturally derived or recombinant products expressed in cells, such as, e.g., proteins, glycoproteins, fusion proteins, growth factors, vaccines, blood factors, thrombolytic agents, hormones, interferons, interleukin based products, monospecific (e.g., monoclonal) antibodies, therapeutic enzymes. Some biologics are approved under a “Biologics License Application” or “BLA”, under section 351(a) of the Public Health Service (PHS) Act, whereas biosimilar and interchangeable biologics referencing a BLA as a reference product are licensed under section 351(k) of the PHS Act. Section 351 of the PHS Act is codified as 42 U.S.C. 262. Other biologics may be approved under section 505(b)(1) of the Federal Food and Cosmetic Act, or as abbreviated applications under sections 505(b)(2) and 505(j) of the Hatch Waxman Act, wherein section 505 is codified 21 U.S.C. 355.

As used herein, “approval” refers to a procedure by which a regulatory entity, e.g., the FDA or EMEA, approves a candidate for therapeutic or diagnostic use in humans or animals. As used herein, a “primary approval process” is an approval process which does not refer to a previously approved protein, e.g., it does not require that the protein being approved have structural or functional similarity to a previously approved protein, e.g., a previously approved protein having the same primary amino acid sequence or a primary amino acid sequence that differs by no more than 1, 2, 3, 4, 5, or 10 residues or that has 98% or more sequence identity. In embodiments the primary approval process is one in which the applicant does not rely, for approval, on data, e.g., clinical data, from a previously approved product. Exemplary primary approval processes include, in the U.S., a Biologics License Application (BLA), or supplemental Biologics License Application (sBLA), a New Drug Application (NDA) under 505(b)(1) of the Federal Food and Cosmetic Act, and in Europe an approval in accordance with the provisions of Article 8(3) of the European Directive 2001/83/EC, or an analogous proceeding in other countries or jurisdictions. As used herein, a “secondary approval process” is an approval process that refers to clinical data for a previously approved product. In embodiments, a secondary approval requires that the product being approved have structural or functional similarity to a previously approved product, e.g., a previously approved protein having the same primary amino acid sequence or a primary amino acid sequence that differs by no more than 1, 2, 3, 4, 5, or 10 amino acid residues or that has at least 98%, 99% or more (100%) sequence identity. In embodiments a secondary approval process is one in which the applicant relies, for approval, on clinical data from a previously approved product. Exemplary secondary approval processes include, in the U.S., an approval under 351(k) of the Public Health Service Act or under section 505(j) or 505(b)(2) of the Hatch Waxman Act and in Europe, an application in accordance with the provisions of Article 10, e.g., Article 10(4), of the European Directive 2001/83/EC, or an analogous proceeding in other countries or jurisdictions.

As used herein, a “target protein” is any protein of interest to which interaction and/or comparison with a second or “test” protein is desired. An exemplary target protein is an antibody, e.g., a CDR-grafted, humanized or human antibody. Other target proteins include glycoproteins, cytokines, hematopoietic proteins, soluble receptor fragments, growth factors, and glycoprotein conjugates (e.g., Fc fusion proteins). In some embodiments, provided methods are useful for identifying, screening, and/or characterizing binding partners for a target protein. In some embodiments, provided methods are useful for characterizing the similarity between a test protein and a target protein. In some embodiments, a target protein is a commercially available, or approved, biologic that defines or provides the basis against which a test protein is measured or evaluated. In embodiments a target protein is commercially available for therapeutic use in humans or animals. In embodiments a target protein was approved for use in humans or animals by a primary approval process. In embodiments a target protein is a reference listed drug for a secondary approval process. Exemplary target proteins include those described herein.

An “MS signal”, as used herein, refers to one or more signals or representations obtained from MS and associated with presence of one or more chemical compounds and/or structural characteristics and/or peptides. In some embodiments, an MS signal is a peak, or point therein, in an MS spectrum. In some embodiments, an MS signal is a plurality of peaks, or points therein, in an MS spectrum.

As used herein, a “stressor” refers to any agent or condition that induces a shift of a protein and/or protein complex from a first state to a second state. In some embodiments, a stressor can induce a conformational change of a protein, e.g., can induce a change from a first conformation to a second conformation. In some embodiments, a stressor can disrupt interaction sites in a protein complex (e.g., deprotection of protein-protein interaction sites). In some embodiments, a stressor is a label (e.g., a covalent label). In some embodiments, a stressor is an isobaric label. Exemplary isobaric labels include, without limitation, TMTs, iTRAQs, and ICATs. In some embodiments, a stressor is heat, pH, and/or oxidation.

“Tolerable”, as used herein, refers to a range of acceptability for one or more characteristics of a protein complex, such as a site(s) of interaction, strength of interaction(s), similarity to a standard and/or target samples. In some embodiments, tolerable refers to a range of acceptability as determined by mass spectrometry, e.g., for one or more pairs of compared MS signals, such as, for example, MS comparison of a protein complex in two or more states as compared to a desired or determined value and/or MS comparison of test protein and a target protein. In some embodiments, a comparison herein is between an assessment or measure of a value of interest (e.g., variability, site of interaction, strength of interaction, etc.) of a protein complex and a desired or determined value. In some embodiments, a comparison herein is an assessment or measure of a value of interest (e.g., variability, site of interaction, strength of interaction, etc.) between an MS signal of a test protein and an MS signal of a target protein, and such compared MS signals are tolerable if a value of interest between them does not exceed (e.g., as determined using a given statistical method) the value of interest determined of such target protein. In some embodiments, MS signals are determined for multiple distinct batches (e.g., 2, 3, 4, 5, or more batches) of a target protein. In some embodiments, MS signals are determined for a test protein and a target protein using the same MS and stressor (e.g., label or level of label). In some embodiments, MS signals are determined for a test protein in a protein-protein complex. In some embodiments, MS signals are determined for a target protein in a protein complex. In some embodiments, a test protein-protein complex and a target protein-protein complex as assessed using the same MS method and stressor (e.g., label or level of label). In some instances, a comparison is tolerable if it meets a predetermined value (e.g., obtained by assessing multiple batches of target protein). In some instances, comparison of MS signals is performed using a representation.

The term “corresponding peptides”, as used herein, refers to two or more peptides having the same amino acid sequence. In some embodiments, corresponding peptides refer to peptides from different samples of the same protein (e.g., a test protein or a target protein) having the same amino acid sequence. In some embodiments, corresponding peptides refer to peptides from a test protein and a target protein having the same amino acid sequence. For example, a peptide from a test protein and a peptide from a target protein are corresponding peptides if they have the same amino acid sequence.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

### DETAILED DESCRIPTION OF CERTAIN EMBODIMENTS

The present disclosure is based, in part, on the discovery that assessment by mass spectrometry (“MS”) of the behavior of labeled proteins can be used to characterize protein-protein interactions. For example, the present disclosure describes that MS can be used to determine a binding epitope of a protein for its binding partner or ligand, and further, that such techniques may be useful to assess the strength of protein-protein interactions (PPIs) with high resolution.

The underlying functionality of most marketed biotherapeutics can depend, in part, on the specificity and strength of PPIs between a biologic and its protein binding partner(s) as these interactions can induce modulation of downstream pathways to achieve drug efficacy. Consequently, sensitive and high-resolution analytical methodologies are needed to elucidate differences in PPIs, e.g., when developing new drugs, biosimilars, biobetters, antibody-drug conjugates (among others). To characterize the specificity of PPIs, methods such as X-ray crystallography and nuclear magnetic resonance (NMR) have been used to reveal localized binding locations, but are generally limited to complexes that can be crystallized or have a low molecular weight, respectively. Certain mass spectrometry (MS)-based analyses have been used for protein-protein interaction analysis, and may provide valuable information for PPIs when X-ray crystallography and/or NMR are either not available or are not applicable. For example, hydrogen-deuterium exchange (HDX), oxidative foot-printing, and covalent labeling are all MS methodologies that can provide information concerning localized protein structure, dynamics, and protein interactions. However, these previous MS-based techniques have generally been limited by low resolution that leads to incomplete interaction coverage and/or poor sensitivity for detecting small differences between samples. To measure the strength of PPIs, techniques such as surface plasmon resonance (SPR) are most often employed. However, SPR requires immobilization of a protein onto a solid support and often suffers from poor ruggedness and robustness. Moreover, SPR only provides global measurements of protein interaction strength. That is, an SPR signal is the culmination of all binding site affinities; thus, when making sample comparisons, differences in localized protein-protein interactions may get averaged out and go undetected. Therefore, there remains a need for methodologies that can characterize the specificity and strength of protein-protein interactions accurately and sensitively.

Described herein are methods that use high amounts of label to purposely denature protein complexes, resulting in decreased labeling protection at protein-protein binding interfaces. When combined with liquid chromatography such as, for example, tandem mass spectrometry (LC-MS/MS) analysis, these methods can yield high resolution (e.g., high labeling sequence coverage) to improve assessment of local binding sites. Moreover, in some instances, these methods may also provide a measure of protein-protein interaction strength, for example, stronger interaction sites will be less prone to interface deprotection from the label. In some embodiments, methods of the present disclosure can use isobaric tandem mass tags (TMTs) as a covalent label for sample multiplexing, which can be applied to differentiate localized PPIs between related biotherapeutics and their functionally relevant target proteins (i.e., binding protein partners). In some instances, PPIs of a protein complex can be characterized, for example, determination of an amino acid sequence of an interaction site and/or strength of a PPI at a particular site. In some embodiments, PPIs of a protein complex can be compared to a predetermined value. In some embodiments, provided methods can be used to identify and/or screen for new protein therapeutics. For example, provided methods can be used to determine if a test protein has suitable binding characteristics with a protein of interest (e.g., binds at an epitope of interest and/or if with a particular strength of interaction at a relevant site). In some embodiments, provided methods can be used to analyze if a protein has suitable binding characteristics as a therapeutic (e.g., as part of a release test).

In some instances, PPIs of a test protein can be compared to corresponding PPIs of a target protein in order to assess biosimilarity. In some embodiments, the present disclosure provides strategies to assess changes in protein-protein interactions (e.g., functional implications) of intentional and/or unforeseen protein modifications. Such assessments can be used, e.g., to evaluate biosimilarity of a protein (e.g., an antibody or Fc-fusion protein) to a target protein (e.g., a target antibody or target Fc-fusion protein), e.g., during one or more stages of process development and/or production of a biosimilar product.

### Analysis Methods

The present disclosure encompasses a recognition that treating or exposing a protein complex to a stressor (e.g., high concentration of label, heat, oxidation, etc.) can induce a conformation shift from a first state to a second state, which can, for example, alter or disrupt associations between proteins in complex. In some embodiments, labeling of a protein complex with high concentrations of label (e.g., an isobaric label) can induce a shift from a first state to a second state, for example, disrupting association between a protein and its binding partner. While associated, sites of interaction between a protein and its binding partner are generally protected from (i.e., inaccessible to) labeling. Disrupting interactions between proteins in a complex, for example, by inducing a conformational shift from a first state to a second state, can expose previously protected sites of interaction. Thus, a protein complex can be differentially labeled in a first and second state (e.g., a second state may be labeled at sites that were inaccessible to label in the first state). In some embodiments, a protein complex is exposed to 2, 3, 4, 5, 6, or more different levels of label, where each level of label corresponds to a different state of the protein complex. Increasing level of label can increasingly disrupt sites of interaction (e.g., expose previously protected sites). In some embodiments, at least one level of label is sufficient to disassociate the proteins in the protein complex.

In some instances, binding characteristics of a protein are assessed by performing MS on a protein complex (e.g., a complex comprising at least two different proteins). In some embodiments, methods of the present disclosure can be used to determine local binding sites of a protein complex (e.g., amino acid residues involved in protein-protein interactions). In some embodiments, methods of the present disclosure can be used to measure protein-protein interaction strength. In some embodiments, methods of the present disclosure can be used to measure strength of local protein-protein interactions (e.g., strength of an interaction at particular sites).

In some embodiments, a level of one or more peptides from a labeled protein complex in a first state is determined by MS and is compared with levels of one or more corresponding peptides from a labeled protein complex in a second state (e.g., a state exposed to a stressor). In some embodiments, a level of peptide from a labeled protein complex determined by MS for a protein complex in more than two different states, e.g., 3, 4, 5, 6, 7, 8, 9, 10 or more states are analyzed. In some embodiments, 3, 4, 5, 6, 7, 8, 9, 10 or more states correspond with 3, 4, 5, 6, 7, 8, 9, 10, or more different levels of a stressor, respectively. In some embodiments, a stressor is a label (e.g., an isobaric, e.g., TMT label). In some embodiments, a level of one or more peptides from a labeled protein complex exposed to a first concentration of label is determined by MS and is compared with levels of one or more corresponding peptides from a labeled protein complex exposed to a second concentration of label. In some embodiments, a level of peptide from a labeled protein complex determined by MS for a protein complex exposed to 2, 3, 4, 5, 6, 7, 8, 9, 10 or more concentrations of label.

In some embodiments, a level of one or more peptides from a labeled test protein complex (e.g., labeled with a first label) is determined by MS and is compared with a level of one or more corresponding peptides from a labeled target protein complex (e.g., labeled with a second label), and a difference in the peptide levels are determined, e.g., to assess similarity of binding interactions between a target and test protein-protein complex. In some instances, a plurality of peptides labeled with the first label are compared to a plurality of corresponding peptides labeled with the second label.

In some embodiments, a level of one or more labeled peptides from a labeled protein complex is determined by MS and is compared with levels of one or more corresponding peptides from an isolated protein binding partner (i.e., not in a complex). For example, a level of one or more labeled peptides from a labeled protein complex that includes a Fc-containing protein and its antigen or ligand is compared with a level of one or more labeled peptides from a labeled antigen or ligand. In some embodiments, labeled peptides are determined by MS for a protein complex in a first state and a second state. In some embodiments, labeled peptides are also determined for a labeled antigen or ligand in at least a first state and a second state.

MS analysis of one or more labeled peptides of proteins and/or protein complexes in stressed and/or unstressed states can also be used to, e.g., determine a sequence of interaction between proteins in a complex (e.g., an antibody/antigen binding epitope). For example, in some embodiments, peptides from an antigen or ligand can be analyzed by MS to determine amounts of different labeled peptides when the antigen or ligand is part of a complex in a first state and/or a second state. In some embodiments, labeled peptides from an antigen or ligand are analyzed by MS for test protein complex and for a target protein complex comprising the antigen or ligand.

In some embodiments, MS analysis of one or more labeled peptides of proteins and/or protein complexes can be used to determine localized binding strength between proteins in a complex at particular binding sites. For example, in some embodiments, a protein complex may be exposed to a concentration gradient of stressor (e.g., label) and labeled peptides of an antigen or ligand that is part of a protein complex are analyzed by MS. In some embodiments, a relative amount of a label peptide correlates with the strength of binding at that particular site. In some embodiments, strength of binding is determined at 1, 2, 3, 4, 5, 6 or more particular binding sites.

Methods described herein utilize mass spectrometry (MS). Mass spectrometry obtains molecular weight and structural information on chemical compounds by ionizing the molecules and measuring either their time-of-flight or the response of the molecular trajectories to electric and/or magnetic fields. The methods of the present disclosure can employ conventional mass spectrometry techniques known to those of skill in the art, and any known MS method can be adapted for use in methods of the disclosure. Exemplary MS methods include, but are not limited to, tandem MS (MS/MS), LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Such methods are described in, e.g., Pitt, Clin. Biochem. Rev. 30:19-34 (2009). Mass spectrometers that can be used in methods of the present disclosure are known in the art and are commercially available from, e.g., Agilent Inc., Bruker Corporation, and Thermo Scientific.

Labels

Methods described herein involve use of labels for MS analysis, and any label known in the art to be useful in MS can be used. In some instances, labels are added (e.g., coupled using an amine-reactive or a thiol-reactive chemistry) to a protein (e.g., via amine or thiol groups of proteins) using known methods. In certain embodiments, a label is a compound that includes a peptide reactive group (e.g., a maleimide moiety, a bromoacetamide moiety, a pyridyldithio moiety, an iodoacetamide moiety, a methanethiosulfonate moiety, an isothiocyanate moiety, and/or an N-hydroxysuccinimide ester moiety).

In some instances, isobaric labels are used. For example, isobaric labels can be used to label amines in proteins and peptides prior to mixing and simultaneous analysis of multiple samples. Isobaric labels are known in the art and generally have the same chemical structure but different isotopic combinations in the mass reporter. Isobaric labels include, for example, Tandem Mass Tags (TMT) and Isobaric tags for relative and absolute quantitation (iTRAQ) (Ross et al., Molecular & Cellular Proteomics, 2004, 3, 1154-1169). TMT and iTRAQ reagents use a pair of mass tags bearing a differential incorporation of carbon and nitrogen isotopes. Two samples are labelled with either the heavy or light tag and then mixed prior to analysis by MS (e.g., LC-MS). A peptide present in both samples will give a pair of precursor ions with the same mass, but with different mass tags after MS/MS. TMT and iTRAQ isobaric labels are commercially available from, e.g., Life Technologies (Carlsbad, Calif.) and Sciex (Framingham, Mass.), respectively.

Other isobaric labels such as isotope-coded affinity tags (ICAT) as well as nonisobaric labels known in the art can be used to compare the higher structure of two protein samples as long as a conformational shift from a first state to a second state, e.g., a protein conformation change, is induced upon labeling. In some instances, a protein (e.g., a test protein and/or a target protein) is subjected to cleavage, e.g., by limited proteolysis and/or chemical cleavage. For example, a protein can be subjected to enzymatic digestion using known enzymes including, but not limited to, trypsin, papain, pepsin, or Lys-C protease. In some instances, chemical cleavage is performed by reducing disulfide bonds in the protein. For example, reduction of disulfide bonds can include contacting a sample with a reducing agent (e.g., dithiothreitol, mercaptoethanol, tributylphosphine, and/or tri(2-carboxyethyl)phosphine hydrochloride).

In some embodiments, a high level of a label is used. For example, in some embodiments, a high level of label is in a concentration range from about 0.1 mM to about 100 mM. In some embodiments, a high level of label is in an amount within a range bounded by a lower limit and an upper limit, the upper limit being larger than the lower limit. In some embodiments, the lower limit may be about 0.1 mM, 0.2 mM, 0.3 mM, 0.4 mM, 0.5 mM, 0.6 mM, 0.7 mM, 0.8 mM, 0.9 mM, 1 mM, 1.2 mM, 1.5 mM, 1.8 mM, 2.0 mM, 2.2 mM, 2.5 mM. 2.8 mM, 3.0 mM, 3.2 mM, 3.5 mM, 3.8 mM, 4.0 mM, 4.2 mM, 4.5 mM, 4.8 mM, 5.0 mM, 5.2 mM, 5.5 mM, 5.8 mM, 6.0 mM, 6.2 mM, 6.5 mM, 6.8 mM, 7.0 mM, 7.2 mM, 7.5 mM, 7.8 mM, 8.0 mM, 8.2 mM, 8.5 mM, 8.8 mM, 9.0 mM, 9.2 mM, 9.5 mM, 9.8 mM, 10.0 mM, 12 mM, 15 mM, 18 mM, 20 mM, 25 mM, 30 mM, or 50 mM. In some embodiments, the upper limit may be about 0.5 mM, 1.0 mM, 2.0 mM, 3.0 mM, 3.2 mM, 3.5 mM, 3.8 mM, 4.0 mM, 4.2 mM, 4.5 mM, 4.8 mM, 5.0 mM, 5.2 mM, 5.5 mM, 5.8 mM, 6.0 mM, 6.2 mM, 6.5 mM, 6.8 mM, 7.0 mM, 7.2 mM, 7.5 mM, 7.8 mM, 8.0 mM, 8.2 mM, 8.5 mM, 8.8 mM, 9.0 mM, 9.2 mM, 9.5 mM, 9.8 mM, 10.0 mM, 12 mM, 15 mM, 18 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, a high level of an isobaric label (e.g., a TMT label) is used.

In some embodiments, a high level of a label is used. In some embodiments, a high level of a label is sufficient to induce a conformational change of a protein complex. In some embodiments, a high level of label is a molar excess of label in a range of about 50 to 100,000 molar excess of label relative to the protein complex (e.g., glycoprotein complex). In some embodiments, a high level of label is in a molar excess that is within a range bounded by a lower limit and an upper limit, the upper limit being larger than the lower limit. In some embodiments, the lower limit may be about 50, 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, or 50000 times molar excess relative to the glycoprotein and/or glycoprotein complex. In some embodiments, the upper limit may be about 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 65000, 70000, 75000, 80000, 85000, 90000, 95000, 100000 times molar excess relative to the protein complex (e.g., glycoprotein complex). In some certain embodiments, a high level of label is in a range of 500 to 1000 molar excess relative to the protein complex (e.g., glycoprotein complex). In some embodiments, a high level of an isobaric label (e.g., a TMT label) is used.

In some embodiments, a mass spectrum of relative abundance of ions with a particular mass/charge over a given range (e.g., 100 to 2000 amu) is obtained. Numerous methods for relating amount of an ion to an amount of a peptide are known to those of ordinary skill in the art. For example, relative abundance of a given ion may be compared to various values (e.g., a table) that can be used to convert a relative abundance to an absolute amount of a peptide. Alternatively, external standards may be run with samples, and a standard curve constructed based on ions generated from such standards. Using a standard curve, relative abundance of a given ion may be converted into an absolute amount of a peptide. Methods of generating and using such standard curves are well known in the art, and one of ordinary skill is capable of selecting an appropriate internal standard.

In some instances, multiple samples of a protein complex (e.g., multiple samples of a test protein in a complex) can be labeled with a plurality of isobaric labels having different mass tags (e.g., 2, 3, 4, 5, 6, 7, 8, 9, 10, 11, 12, 13, 14 or more labels having different mass tags). In some instances, multiple samples of a protein complex and one or more isolated proteins that are part of the complex can be labeled with a plurality of isobaric labels having different mass tags (e.g., 2, 3, 4, 5, 6, 7, 8, 9, 10, 11, 12, 13, 14 or more labels having different mass tags). In some instances, multiple samples of a first protein complex and a second protein complex, wherein the complexes have a common binding partner (e.g., antigen, ligand, etc.) can be labeled with a plurality of isobaric labels having different mass tags (e.g., 2, 3, 4, 5, 6, 7, 8, 9, 10, 11, 12, 13, 14 or more labels having different mass tags).

In some instances, the plurality of isobaric labels is an “x-plex” of TMT labels, such as a duplex, a “sixplex”, a “10-plex” or a “12-plex”. In an exemplary method, a sixplex of TMT labels is used, each label having a different mass (e.g., 126, 127, 128, 129, 130, and 131). For example, each of three samples of a test protein in a first state can be independently labeled with one of three TMT labels (e.g., 126, 127, and 128), and each of three samples of a test protein in a second state can be independently labeled with three different TMT labels (e.g., 129, 130, and 131). Use of such TMT sixplex procedure allows three replicates of a test protein complex in a first state and three replicates of a test protein complex in a second state to be analyzed using a single MS sample preparation and one MS run. Without wishing to be bound by theory, it is believed that because of such multiplexing capability, variability from differences in, e.g., MS ionization, data-dependent peak picking, and/or sample preparation is reduced, improving repeatability and/or robustness.

In some instances, levels of corresponding labeled peptides (e.g., labeled peptides from a protein complex in 2 or more different states, e.g. exposed to two or more different levels of stressor, e.g., exposed to two or more different levels of label) are obtained, identified, assessed, measured, determined and/or quantified. Such levels can be compared to determine a site and/or strength of localized PPI.

In some instances, levels of corresponding labeled peptides (e.g., labeled peptides from a test protein and/or its binding partner and corresponding labeled peptides from a target protein and/or the binding partner) are obtained, identified, assessed, measured, determined and/or quantified. Such levels can be compared to determine a level of similarity of binding characteristics between a test protein and a target protein.

Numerous methods for determining a binding site and/or local strength of binding from relative amounts of labeled peptides are available to those of ordinary skill in the art. For example, relative abundance of a given peptide in a first state may be compared with a given peptide from a protein or protein complex in a second state. In some embodiments, a standard curve may be used to determine convert given abundance of a given peptide into a epitope or binding sequence.

In some instances, proteins and/or protein complexes are labeled to induce a shift in the protein and/or protein complex from a first state to a second state, and are also exposed to one or more additional stressor(s) described herein to induce further conformational changes.

### Applications

In some instances, methods disclosed herein can be used to confirm the identity and/or quality of a protein, e.g., glycoprotein preparation. For example, methods can include assessing preparations (e.g., samples, lots, and/or batches) of a protein, e.g., to confirm whether a test protein qualifies as a target protein, and, optionally, qualifying the test protein as a target protein if qualifying criteria (e.g. predefined qualifying criteria) are met; thereby evaluating, identifying, and/or producing (e.g., manufacturing) a protein product. In some embodiments, provided methods of assessing preparations of a protein (e.g., samples, lots, and/or batches) are useful to for qualifying the protein for release as a protein product, such as determining if qualifying criteria (e.g. predefined qualifying criteria) are met.

In some embodiments, MS analysis of one or more labeled peptides of proteins and/or protein complexes can be used to determine a sequence of interaction between proteins in a complex (e.g., an antibody/antigen binding epitope). In some embodiments, MS analysis of one or more labeled peptides of proteins and/or protein complexes can be used to determine localized binding strength between proteins in a complex at particular binding sites.

Methods of the disclosure have a variety of applications and include, e.g., quality control at different stages of manufacture, analysis of a protein preparation prior to and/or after completion of manufacture (e.g., prior to or after distribution to a fill/finish environment or facility), prior to or after release into commerce (e.g., before distribution to a pharmacy, a caregiver, a patient, or other end-user). In some instances, a protein preparation is a drug substance (an active pharmaceutical ingredient or “API”) or a drug product (an API formulated for use in a subject such as a human patient). In some instances, a protein preparation is from a stage of manufacture or use that is prior to release to care givers or other end-users; prior to packaging into individual dosage forms, such as syringes, pens, vials, or multi-dose vials; prior to determination that the batch can be commercially released, prior to production of a Certificate of Testing, Material Safety Data Sheet (MSDS) or Certificate of Analysis (CofA) of the preparation. In some instances, a protein preparation is from an intermediate step in production, e.g., it is after secretion of a protein from a cell but prior to purification of drug substance.

Evaluations from methods described herein are useful for guiding, controlling or implementing a number of activities or steps in the process of making, distributing, and monitoring and providing for the safe and efficacious use of a protein preparation. Thus, in an embodiment, e.g., responsive to the evaluation, e.g., depending on whether a criterion is met, a decision or step is taken. In some embodiments, a method can further comprise one or both of the decision to take the step and/or carrying out the step itself. For example, the step can comprise one in which the preparation (or another preparation for which the preparation is representative) is: classified; selected; accepted or discarded; released or processed into a drug product; rendered unusable for commercial release, e.g., by labeling it, sequestering it, or destroying it; passed on to a subsequent step in manufacture; reprocessed (e.g., the preparation may undergo a repetition of a previous process step or subjected to a corrective process); formulated, e.g., into drug substance or drug product; combined with another component, e.g., an excipient, buffer or diluent; disposed into a container; divided into smaller aliquots, e.g., unit doses, or multi-dose containers; combined with another preparation of the protein; packaged; shipped; moved to a different location; combined with another element to form a kit; combined, e.g., placed into a package with a delivery device, diluent, or package insert; released into commerce; sold or offered for sale; delivered to a care giver or other end-user; or administered to a subject. For example, based on the result of a determination or whether one or more subject entities is present, or upon comparison to a reference standard, the batch from which the preparation is taken can be processed, e.g., as just described.

Methods described herein may include making a decision: (a) as to whether a protein preparation may be formulated into drug substance or drug product; (b) as to whether a protein preparation may be reprocessed (e.g., the preparation may undergo a repetition of a previous process step); and/or (c) that the protein preparation is not suitable for formulation into drug substance or drug product. In some instances, methods comprise: formulating as referred to in step (a), reprocessing as referred to in step (b), or rendering the preparation unusable for commercial release, e.g., by labeling it or destroying it, as referred to in step (c).

In some embodiments, such decisions can be made by determining one or more sites of interaction of a test protein and a protein binding partner (e.g., by comparing a test MS signal of a labeled test protein-protein complex in a first state and a second state). For example, in some embodiments, if a site of interaction is tolerable, a batch of test protein drug substance may be processed into a drug product.

In some embodiments, such decisions can be made by determining the strength of binding between a test protein and a protein binding partner. For example, in some embodiments, if the strength of binding between a test protein and a protein binding partner (e.g., total binding and/or binding strength at one or more sites of interaction), a batch of test protein drug substance may be processed into a drug product. In some embodiments, the binding strength is considered tolerable when it meets a predetermined value. In some embodiments, the binding strength is considered tolerable when it differs by no more than 30%, 20% or 10% from a desired and/or determined binding strength of a protein to the protein binding partner (e.g., total binding and/or binding strength at the particular site).

In some embodiments, processing into a drug product can include one or more steps of: formulating a test protein; combining a test protein with a second component, e.g., an excipient or buffer; changing the concentration of a test protein in a preparation; lyophilizing a test protein; combining a first and second aliquot of a test protein to provide a third, larger, aliquot; dividing a test protein into smaller aliquots; disposing a test protein into a container, e.g., a gas or liquid tight container; packaging a test protein; associating a container comprising a test protein with a label (e.g., labeling); shipping or moving a test protein to a different location.

In some embodiments, if a site of interaction is not tolerable, an alternative action may be taken, such as, for example, disposing of a test protein, classifying a test protein for disposal, labeling a test protein for disposal, and/or reprocessing a test protein. In some embodiments, if the strength of binding between a test protein and a protein binding partner is not tolerable, an alternative action may be taken, such as, for example, disposing of a test protein, classifying a test protein for disposal, labeling a test protein for disposal, and/or reprocessing a test protein.

In an exemplary method, MS is used to assess the similarity of a test biologic to a reference biologic that is approved under a BLA. In an exemplary method, a reference complex including a reference biologic with a binding partner and a test complex including a test biologic with the same binding partner, can each be separately labeled with amine-reactive isobaric labels, which upon dissociation (e.g., by MS/MS) yield reporter ions of different mass. Labeled protein complexes are sequentially mixed about 1:1, denatured, reduced, alkylated, enzymatically digested, and analyzed by LC-MS/MS. Peptides are identified by database searching MS/MS spectra, and reporter ion ratios are used to calculate fold changes (i.e., localized structural deviations) for each labeled peptide. While some methods described herein recite a particular order of steps (e.g., labeling, denaturing, reducing, alkylating, and/or digesting), in some instances, one or more steps can be performed in a different order.

### Protein Complexes

Methods described herein can be used to make, characterize, and/or evaluate interactions between proteins in a protein complex (e.g., a protein-protein complex comprising two or more proteins). In some embodiments, provided methods may be useful for identifying and/or screening for protein binding partners with qualifying characteristics.

In some embodiments, methods can be used to make, characterize, and/or evaluate a test protein preparation, e.g., a test biologic preparation. In some embodiments, a test protein is a test biologic being evaluated for similarity to a target protein, e.g., a target biologic. A test biologic may or may not be commercially available. In some embodiments, a test biologic is not commercially available for therapeutic use in humans or animals. In some embodiments, a test biologic has not been approved for therapeutic or diagnostic use in humans or animals. In some embodiments, a test biologic has been approved, e.g., under a secondary approval process, for therapeutic or diagnostic use in humans or animals. In some embodiments, a test protein (e.g., test biologic) has the same primary amino acid sequence as a target protein (e.g., target biologic) or will differ by no more than 1, 2, 3, 4, 5, 10, 15, 20, 25, 30 residues and/or has at least 90, 95, 98, 99% or is identical to a target protein sequence (e.g., target biologic sequence). The terms the “same primary amino acid sequence”, “a primary amino acid sequence that differs by no more than 1, 2, 3, 4, 5, 10, 15, 20, 25, or 30 residues”, “sequences that have at least 98% or more sequence identity”, or similar terms, relate to level of identity between a primary amino acid sequence, e.g., of first protein, e.g., a test protein, and a primary amino acid sequence, e.g., of second protein, e.g., a target protein. In some embodiments, a protein preparation or product includes amino acid variants, e.g., species that differ at terminal residues, e.g., at one or two terminal residues. In some embodiments of such cases, sequence identity compared is the identity between the primary amino acid sequence of the most abundant (e.g., most abundant active) species in each of the products being compared. In some embodiments, sequence identity refers to the amino acid sequence encoded by a nucleic acid that can be used to make the product.

Nonlimiting, exemplary target proteins can include abatacept (Orencia®, Bristol-Myers Squibb), abciximab (ReoPro®, Roche), adalimumab (Humira®, Bristol-Myers Squibb), aflibercept (Eylea®, Regeneron Pharmaceuticals), alefacept (Amevive®, Astellas Pharma), alemtuzumab (Campath®, Genzyme/Bayer), basiliximab (Simulect®, Novartis), belatacept (Nulojix®, Bristol-Myers Squibb), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denileukin diftitox (Ontak®, Eisai), denosumab (Prolia®, Amgen; Xgeva®, Amgen), eculizumab (Soliris®, Alexion Pharmaceuticals), efalizumab (Raptiva®, Genentech), etanercept (Enbrel®, Amgen-Pfizer), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rilonacept (Arcalyst®, Regeneron Pharmaceuticals), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and ustekinumab (Stelara®, Janssen).

Antibodies

In some instances, test proteins and target proteins described herein are antibodies. As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)2, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda. In some embodiments, an antibody includes an Fc region. In some embodiments, an antibody is a therapeutic antibody.

As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology.

In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

Antibodies or fragments thereof can be produced by any method known in the art for synthesizing antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

As used herein, the term “antibody agent” refers to an agent that specifically binds to a particular antigen. In some embodiments, the term encompasses any polypeptide or polypeptide complex that includes immunoglobulin structural elements sufficient to confer specific binding. In some embodiments, an antibody agent is or comprises a polypeptide whose amino acid sequence includes one or more structural elements recognized by those skilled in the art as a complementarity determining region (CDR); in some embodiments, an antibody agent is or comprises a polypeptide whose amino acid sequence includes at least one CDR (e.g., at least one heavy chain CDR and/or at least one light chain CDR) that is substantially identical to one found in a reference antibody. In some embodiments, an included CDR is substantially identical to a reference CDR in that it is either identical in sequence or contains between 1-5 amino acid substitutions as compared with the reference CDR. In some embodiments, an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 96%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Glycoprotein Conjugates

In some instances, test proteins and target proteins are glycoprotein conjugates (e.g., Fc regions or Fc fragments containing one or more N-glycosylation sites thereof that are conjugated or fused to one or more heterologous moieties). Heterologous moieties include, but are not limited to, peptides, polypeptides, proteins, fusion proteins, nucleic acid molecules, small molecules, mimetic agents, synthetic drugs, inorganic molecules, and organic molecules. In some instances, a glycoprotein conjugate is a fusion protein that comprises a peptide, polypeptide, protein scaffold, scFv, dsFv, diabody, Tandab, or an antibody mimetic fused to an Fc region, such as a glycosylated Fc region. A fusion protein can include a linker region connecting an Fc region to a heterologous moiety (see, e.g., Hallewell et al. (1989), J. Biol. Chem. 264, 5260-5268; Alfthan et al. (1995), Protein Eng. 8, 725-731; Robinson & Sauer (1996)).

### Recombinant Gene Expression

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

In some embodiments, a protein or protein-protein complex described herein is produced using recombinant methods. Recombinant expression of a gene, such as a gene encoding a polypeptide, such as an antibody described herein, can include construction of an expression vector containing a polynucleotide that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and transfected cells can then be cultured by conventional techniques to produce polypeptide.

A variety of host expression vector systems can be used (see, e.g., U.S. Pat. No. 5,807,715). Such host-expression systems can be used to produce polypeptides and, where desired, subsequently purified. Such host expression systems include microorganisms such as bacteria (e.g., E. coli and B. subtilis) transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g. Ti plasmid) containing polypeptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For bacterial systems, a number of expression vectors can be used, including, but not limited to, the E. coli expression vector pUR278 (Ruther et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 8 1:355-359). The efficiency of expression can be enhanced by inclusion of appropriate transcription enhancer elements, transcription terminators, etc. (see, e.g., Bittner et al., 1987, Methods in Enzymol. 153:516-544).

In addition, a host cell strain can be chosen that modulates expression of inserted sequences, or modifies and processes the gene product in the specific fashion desired. Different host cells have characteristic and specific mechanisms for post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, but are not limited to, CHO, VERY, BHK, HeLa, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a protein or protein-protein complex described herein been produced by recombinant expression, it may be purified by any method known in the art for purification, for example, by chromatography (e.g., ion exchange, affinity, and sizing column chromatography), centrifugation, differential solubility, or by any other standard technique for purification of proteins. For example, an antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a glycoprotein can be fused to heterologous polypeptide sequences to facilitate purification. Glycoproteins having desired sugar chains can be separated with a lectin column by methods known in the art (see, e.g., WO 02/30954).

### Pharmaceutical Compositions

A protein (e.g., an antibody) or protein complex, produced or analyzed using any of the methods described herein can be incorporated into a pharmaceutical composition. Such a pharmaceutical composition may be useful in the prevention and/or treatment of diseases. Pharmaceutical compositions comprising a polypeptide (e.g., an antibody) can be formulated by methods known to those skilled in the art (see, e.g., Remington's Pharmaceutical Sciences, 20th Ed., Lippincott Williams & Wilkins, 2000). The pharmaceutical composition can be administered parenterally in the form of an injectable formulation comprising a sterile solution or suspension in water or another pharmaceutically acceptable liquid. For example, the pharmaceutical composition can be formulated by suitably combining the polypeptide with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

A suitable means of administration can be selected based on the age and condition of the patient. A single dose of the pharmaceutical composition containing a polypeptide (e.g., antibody) can be selected from a range of 0.001 mg/kg of body weight to 1,000 mg/kg of body weight. On the other hand, a dose can be selected in the range of 0.001 mg/kg of body weight to 100,000 mg/kg of body weight, but the present disclosure is not limited to such ranges. Dose and method of administration varies depending on the weight, age, condition, and the like of the patient, and can be suitably selected as needed by those skilled in the art.

The disclosure is further illustrated by the following examples. The examples are provided for illustrative purposes only. They are not to be construed as limiting the scope or content of the disclosure in any way.

### EXAMPLES

### Example 1: Characterization of a Model Antibody-Antigen Complex

This example describes characterization of a model antibody-antigen complex using covalent labeling denaturation methodology. A model monoclonal antibody, IgG1(a), that binds to a model antigen, TNFα, was characterized. Protein-protein interaction sites of an exemplary TNFα/IgG1(a) complex have been mapped by x-ray crystallography (Hu, S. et al., Journal of Biological Chemistry, 2013, 38, 27059-27067). Effects of increasing label concentration on method resolution and the extent of denaturation based on label concentration was assessed. Model antigen (TNFα) alone (i.e., no antibody bound) and model antigen-antibody TNFα/IgG1(a) complex were labeled by isobaric tagging with commercially available Tandem Mass Tags (“TMT”) with reporter ions at m/z 126 and 127 (Life Technologies, Carlsbad, Calif.), respectively, at the following concentrations: 0.3 mM, 0.5 mM, 1.1 mM, 2.7, mM, 5.3 mM, and 9.8 mM. After quenching the labeling reaction, TNFα and the TNFα/IgG1(a) complexes were mixed 1:1 at each TMT concentration, denatured, reduced, alkylated, enzymatically digested, and analyzed by LC-MS/MS. TNFα peptides were identified by database searching MS/MS spectra, and the TMT 127/126 reporter ion ratios were used to calculate fold changes (i.e., localized structural deviations) for each TNFα-labeled peptide. A fold change of approximately 1 indicates equivalence between the two samples for a given TMT labeled peptide (i.e., the peptide is not involved in a protein-protein interaction) and a negative fold change indicates a protein-protein interaction in the TNFα/IgG1(a) complex that is protected from the label. Without wishing to be bound by theory, positive fold changes may result from label-induced unfolding of TNFα when in complex with the antibody, an experimental artifact that is most apparent at high TMT concentrations.

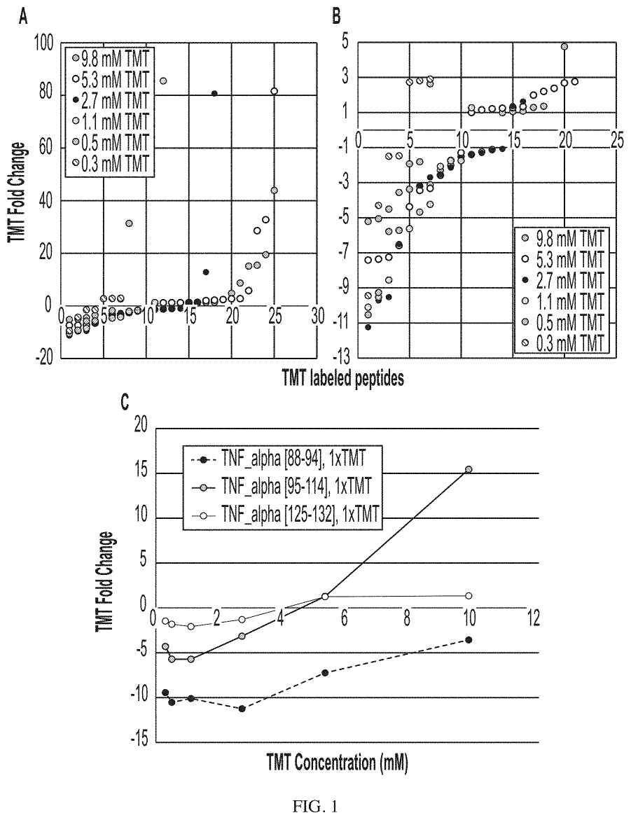

As shown in FIG. 1 panel (A), labeling with increasing amounts of label was found to increase resolution (i.e., number of TMT labeled amino acid residues and peptides) for differentiating localized binding sites of a model complex of an antibody agent and antigen (i.e., TNFα/IgG1(a) complex). FIG. 1 panel (B) depicts labeling of a truncated version of (A), highlighting TMT peptides with negative fold changes, which indicate more localized protection during covalent labeling of the complex versus TNFα alone. Thus, high concentration of label promotes characterization of all or nearly all binding sites.

TMT reagents most readily modify lysine residues and protein N-termini, but can also modify tyrosines, serines, threonines, and histidines at high label concentrations. With this model complex and conditions, a majority of peptides identified only at the highest label concentrations were a result of tyrosine and serine modifications as well as multiple lysine modifications on a single peptide. However, at these higher TMT labeling conditions, fold changes often become more positive from decreased localized protection, which may be the result of protein denaturation.

FIG. 1 panel (C) depicts how increasing the amount of label denatures the model complex, decreases localized protection, and thus fold changes become more positive during the TMT reaction. Three examples of TMT-labeled model TNFα peptides are shown that have the same TMT peptide identified at all labeling concentrations tested. The two peptides at residues 88-94 and 95-114 of a model TNFα antigen are known to be involved in the binding sites of the TNFα/IgG1(a) complex. A peptide spanning residues 125-132 is not directly involved in protein-protein binding, but is 2 residues adjacent to a known interface. For the two peptides involved in the PPIs of the model complex, fold changes become considerably more positive at increasing TMT concentrations indicating a decrease in labeling protection at the PPI binding site. While the other peptide, TNFα (125-132), exhibited some decrease in labeling protection, the fold changes were near one since the peptide is not directly involved in the protein-protein contact. These results show that the highest TMT concentrations yield the most labeled peptide sequence coverage. Thus, high sequence coverage using high concentrations of label can provide holistic assessment of localized higher order. However, high labeling conditions can often result in a decrease in labeling protection at protein-protein binding sites, and thus confound the method's ability to map the epitope.

To assess which TMT concentrations induce protein complex denaturation, the labeling kinetics of the TMT reaction were monitored by generating dose-response curves as described in detail previously (Zhou, Y. and Vachet, R. W., Analytical Chemistry, 2013, 85, 9664-9670). This procedure can sensitively detect label-induced structural perturbations at localized protein sites since the amount of covalent attachment between a TMT reagent and protein will scale linearly with reagent concentration. A deviation in linearity denotes an alteration in the native protein conformation. Dose-response curves were generated by reacting the model TNFα/IgG1(a) complex with TMT reagent using the same label concentrations used for the experiment described above (0.3 mM, 0.5 mM, 1.1 mM, 2.7, mM 5.3 mM, and 9.8 mM), and LC-MS/MS analysis was subsequently performed. Precursor areas were calculated from the resulting TMT-modified TNFα peptide identifications, and plots were generated using the following equation:

$$ln ( \frac{Area of unmodified}{Area of unmodified + Area of modified} ) vs [ TMT ]$$

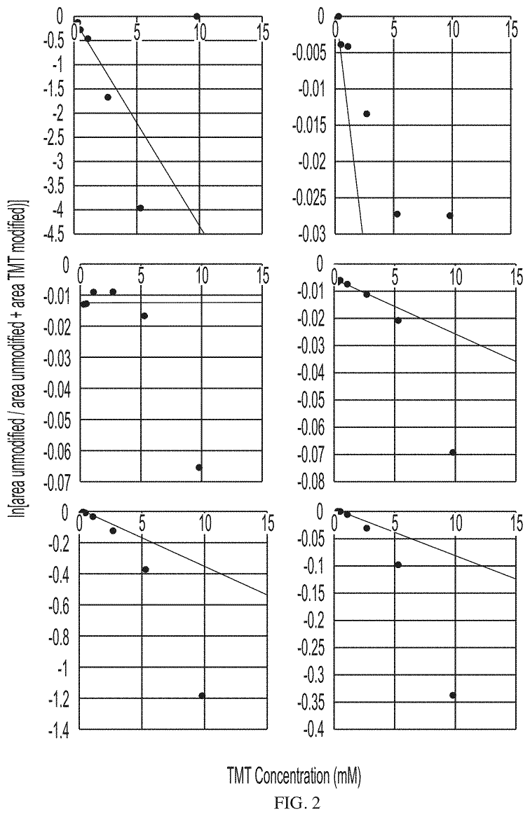

Dose-response plots for all unique TMT-labeled peptides for an antigen of a model antibody-antigen complex (i.e., TNFα/IgG1(a) complex) that had unmodified and modified area counts are shown in FIG. 2. Unique TMT-labeled peptides that had unmodified and modified area counts that were detectable for at least four of the six concentrations are shown. A straight line was drawn through the points to emphasize any deviations from linearity. The majority of the TMT-modified TNFα peptides demonstrated no significant structural perturbations at the lowest concentrations (0.3 mM, 0.5 mM, and 1.1 mM) as these points mostly scaled linearly. However, deviations from linearity were detected for the model TNFα/IgG1(a) complex reactions using 2.7 mM TMT, but not for all peptides. Substantial deviations from linearity were observed for the two highest concentrations (5.3 mM and 9.8 mM) for essentially every TMT peptide. This data further demonstrates that high label amounts can significantly denature a model protein complex.

### Example 2: Characterization of a Model Antibody-Antigen Complex Using a TMT 6plex Procedure

This example describes a further assessment of localized covalent labeling denaturation of a model antibody-antigen complex using TMT sixplex labels. Epitope sequence coverage and fold change effects from using high labeling conditions were determined. As the highest TMT concentration (9.8 mM) tested above did not significantly increase the number of identified TMT-modified peptides from a model complex as compared to the next highest concentration (5.3 mM) (FIG. 1 panel (A)), a TMT concentration of 5.3 mM was used for the following experiments. This concentration yields an approximately 1000 molar excess of label per protein. TMT 126, 127, and 128 were utilized for labeling three replicate aliquots of model antigen TNFα and TMT 129, 130, and 131 for labeling three replicates of TNFα/IgG1(a) aliquots. Samples were mixed 1:1:1:1:1:1 after quenching, and were then analyzed by LC-MS/MS.

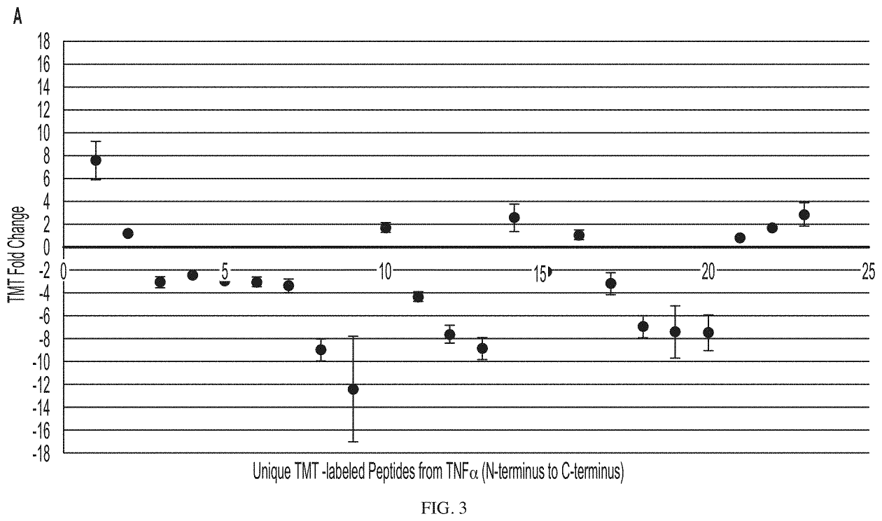

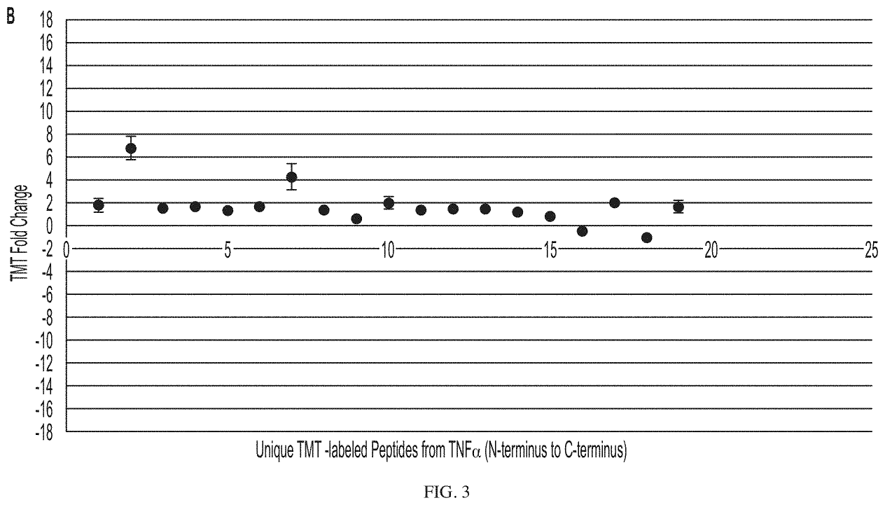

FIG. 3 panel (A) depicts a plot of fold change versus unique TMT-modified TNFα peptides (arranged from N- to C-terminus) from TMTsixplex labeling of TNFα alone versus the TNFα/IgG1(a) complex. Fold changes were calculated from the average of the 129/126, 130/127, and 131/128 reporter ions (decimal fold changes were converted to negative reciprocals before averaging). Higher positive and negative fold changes indicate more localized protection during covalent labeling denaturation for TNFα alone and TNFα/IgG1 complex, respectively. A fold change near one indicates equivalence between the two samples for a given TMT labeled peptide.

Several locations on model antigen TNFα exhibited significant label protection (i.e., negative fold changes) after labeling with 5.3 mM TMT. For example, TMT peptides were considered significantly different between samples if: |fold change|−|error|>2. Therapeutic proteins generally bind their target protein(s) at multiple sites with high affinities, and as these results demonstrate, many of the epitope sites in the TNFα/IgG1(a) complex are resistant to complete label-induced deprotection. Weaker PPIs in stress-induced aggregates have previously been shown to completely lose labeling protection under similar TMT conditions (Madsen, J. A. et al., Analytical Chemistry, 2016, 88, 2678-2488). As a control, a different therapeutic IgG1 that does not bind TNFα was analyzed per the same procedure. This nonspecific monoclonal antibody showed no significant negative fold changes across the TNFα sequence (FIG. 3, panel B). This supports that a covalent labeling denaturation procedure as describe herein does not generate any false artifacts that would suggest erroneous binding locations.

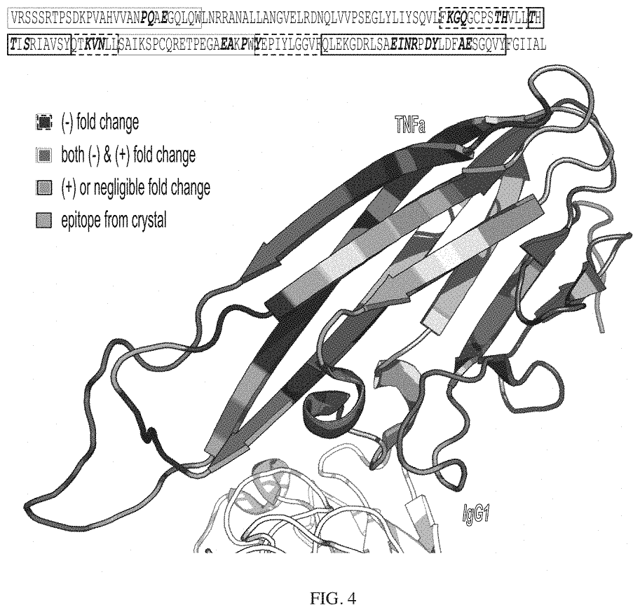

FIG. 4 shows TMT-labeled peptide sequence coverage for TNFα (top) and structural assessment from covalent labeling denaturation of TNFα alone versus TNFα/IgG1(a) complex. Overall, covalent labeling denaturation using TMT sixplex labels yielded high labeling coverage and was able to generate a fold change measurement on all known binding regions of TNFα (known binding sites from x-ray crystallography analysis were highlighted in yellow). Areas that were not labeled generally had a series of amino acids that were unreactive to the TMT label and/or were highly buried in the interior of the TNFα structure; these regions were outside of all interaction sites. Interestingly, some parts of the TNFα epitope regions exhibited negligible fold changes of near one (purple highlights) while other known interaction sites showed a blend of positive and negative fold changes (red highlights). The variation in fold changes across the various protein-protein contact sites can be attributed to several factors. First, some TMT-labeled peptides are fairly large and the modified residues could be significantly out of the interaction protection of certain epitope sites. The use of high label concentrations increases sequence coverage and helps to significantly circumvent this issue; however, some interaction sites can still be challenging to label. Furthermore, label-induced denaturation of complex at high reagent concentrations often reduces the degree of labeling protection as previously shown. Modified residues that have less protection and/or weaker binding strength, therefore, may have negligible or even positive fold changes due to label denaturation. Peptides that yielded both positive and negative fold changes usually had various degrees of labeling identified for the same peptide species. These results further indicate that increased method resolution and thus more comprehensive localized PPI characterization comes at the expense of certain binding site determinations.

Covalent labeling has traditionally been performed at low reagent concentrations at least in part because high reagent loads lead to ambiguities in native protein conformations. However, functional implications of intentional and/or unforeseen protein modification are often assessed as a comparison during the development of therapeutic proteins (whether that be a biosimilar compared to the original innovator material, an antibody-drug conjugate (ADC) compared to its naked mAb, biologics stressed at different conditions, among others). Accordingly, it is often more important for a given analytical technique to have a high differentiating capability that can detect and/or predict potential alterations in drug function than to measure exact native quaternary structural conformation. The present disclosure provides the insight that covalent labeling denaturation methods could be well suited for detecting and/or quantifying variations in protein-protein interaction comparisons. For example, given that weaker protein-protein interactions may be more prone to effects of label-induced denaturation (i.e., interface deprotection from high reagent loads), such methods could not only be used to increase labeling coverage and thus resolution, but may also provide a direct measurement of localized protein interaction strength.

### Example 3: Differentiating Localized Protein-Protein Interactions of a Model Antibody-Antigen Complex

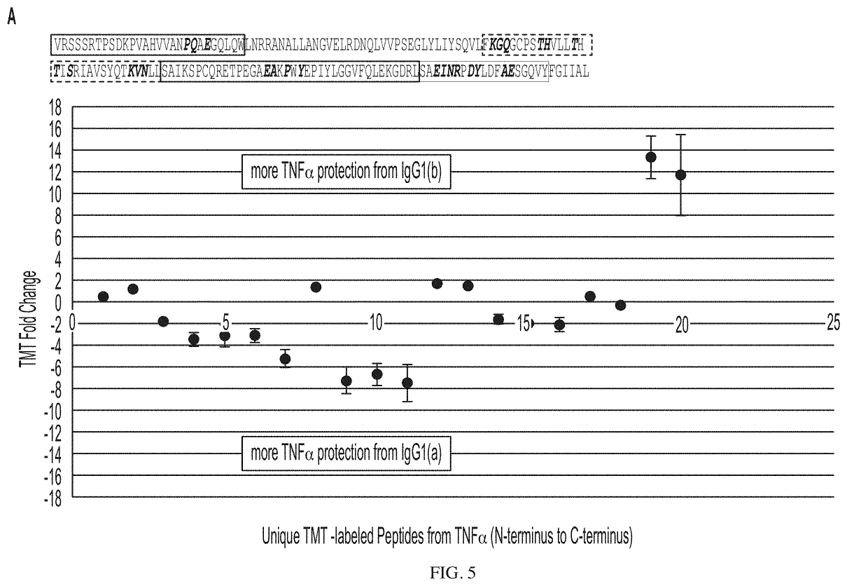

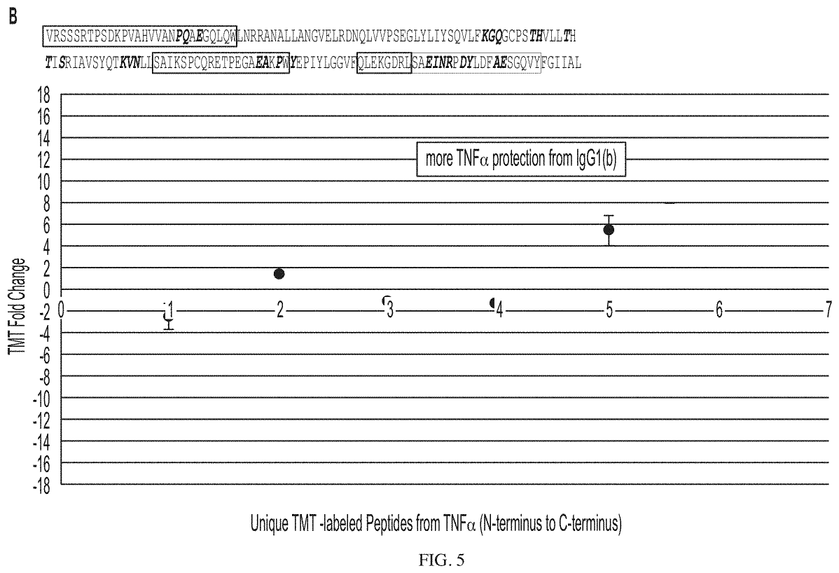

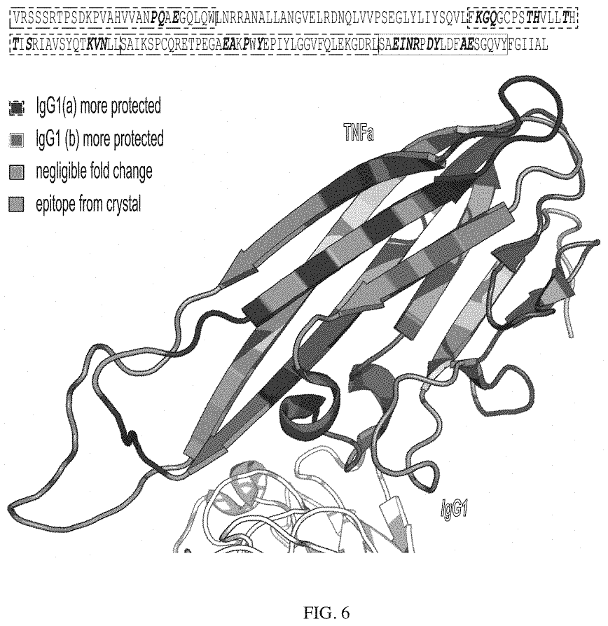

This example describes using covalent labeling denaturation methods to differentiate localized protein-protein interactions between related biotherapeutics and their target proteins. The first comparison was made between the model TNFα/IgG1(a) complex and another TNFα complex involving IgG1(b), a therapeutic mAb that targets TNFα with a slightly different specificity as compared to IgG1(a). In this experiment, TMT 126, 127, and 128 were reacted with three replicates of TNFα/IgG1(b) aliquots and TMT 129, 130, and 131 were reacted with three replicates of TNFα/IgG1(a) aliquots. The denaturing labeling conditions of 5.3 mM TMT were used for each reaction. FIG. 5 panel A illustrates the TMT-labeled peptide sequence coverage for TNFα (top) and localized structural assessment (bottom) for the PPI differences between the two complexes. Blue highlights in the sequence coverage maps indicate that TNFα was more protected with IgG1(a), red highlights indicate that TNFα was more protected with IgG1(b), and purple highlights specify negligible fold changes between the samples. Yellow letters represent the TNFα/IgG1(a) epitope sites previously reported using crystallography. High TMT-labeled sequence coverage was again observed with all epitope sites covered by at least one TMT-labeled peptide. The results showed that IgG1(a) and IgG1(b) bind TNFα significantly differently at specific localized sections—certain areas yielded more label protection for IgG1(a) versus IgG1(b) while others exhibited the opposite effect. Interestingly, several of the known epitope sites that produced negligible fold changes for TMTsixplex labeling of TNFα alone versus the TNFα/IgG1(a) complex (as previously described), yielded substantial fold changes between the TNFα/IgG1(a) and TNFα/IgG1(b) complexes. The TMT-labeled peptide SAEINRPDYLDFAESGQVY (SEQ ID NO: 1), for example, yielded a fold change of >10, which indicates that TNFα/IgG1(b) was significantly less prone to label-induced protein interaction deprotection as compared to the TNFα/IgG1(a) complex. These results indicate that IgG1(b) binding is likely stronger in that particular TNFα region. For a better visual representation of the data, the results from FIG. 5 panel A have been mapped onto the structure of the TNFα/IgG1(a) complex, and can be seen in FIG. 6.

In contrast, binding analysis methods such as surface plasmon resonance (SPR) are limited at least in part because they can only assess whole complex interactions (i.e., sum of all the interactions between two proteins). To illustrate this difference, binding affinity for a model TNFα/IgG1(a) complex and another TNFα complex involving IgG1(b) was determined by SPR analysis was compared with covalent labeling denaturation LC-MS/MS. For surface plasmon resonance results, K_{D} fold changes were calculated by dividing the appropriate K_{D} values for each set of experiments. Label denaturation results show TMT peptides that passed the following criteria: |fold change|−|error|>2.0. Negative versus positive TMT fold changes indicate that the target protein was more protected from the label (stronger binding) in the complex listed on top versus bottom, respectively, in the far left column for each set of experiments. A summary of the binding affinities as determined by SPR analysis and an exemplary covalent labeling denaturation LC-MS/MS are shown below in Table 1 and Table 2, respectively.

TABLE 1
SPR analysis of a model TNFα/IgG1(a) complex
and a model TNFα/IgG1(b) complex

| | | | | K_{D} Fold |
|---|---|---|---|---|
| Sample | ka (1/Ms) | kd (1/s) | K_{D} (M) | Change |
| TNFα/IgG1(a) | 7.52E+05 | 1.27E−04 | 1.69E−10 | |
| TNFα/IgG1(b) | 4.44E+06 | 2.81E−04 | 6.34E−11 | 2.66 |

TABLE 2
covalent labeling denaturation LC-MS/MS
analysis of a model TNFα/IgG1(a) complex
and a model TNFα/IgG1(b) complex

| | | SEQ ID | # of TMT | TMT Fold |
|---|---|---|---|---|
| Sample | Peptide | NOS | Labels | Change |
| TNFα/IgG1(a) | [L].FKGQGCPSTHVLL.[T] | 2 & 3 | 1 | -3.44 |
| complex | | | | |
| TNFα/IgG1(b) | [L].FKGQGCPSTHVL.[L] | 4 & 5 | 1 | -3.18 |
| complex | | | | |
| | [F].KGQGCPSTHVLL.[T] | 6 & 7 | 1 | -3.08 |
| | [F].KGQGCPSTHVL.[L] | 8 & 9 | 1 | -5.20 |
| | [L].THTISRIAVSY.[Q] | 10 & 11 | 1 | -7.27 |
| | [Y].QTKVNL.[L] | 12 & 13 | 1 | -6.66 |
| | [Y].QTKVNLL.[S] | 14 & 15 | 1 | -7.50 |
| | [L].SAEINRPDYLDF.[A] | 16 & 17 | 1 | 13.35 |
| | [L].SAEINRPDYLDFAESGQVY.[F] | 18 & 19 | 1 | 11.70 |

FIG. 5B shows the outcome of the same experiment as in FIG. 5A with a significantly reduced concentration of label (0.5 mM TMT) per reaction. This labeling amount was determined to be nondenaturing of the model protein complex (See, e.g., FIGS. 1 and 2). Only peptides with the most easily modifiable lysine residues were detected, and only one TMT modification per peptide was observed, which greatly reduced the resolution of the method as shown in the TMT sequence coverage map (FIG. 5 panel B). Many of the protein-protein interaction sites went undetected, and thus an incomplete picture of PPI differences between the two complexes was the consequence. Interestingly, the fold change for the same TMT-labeled peptide SAEINRPDYLDFAESGQVY (SEQ ID NO: 1) was also substantially smaller (less sensitive in detecting the PPI difference) for the nondenaturing versus denaturing conditions, a result we have observed previously when studying tertiary biotherapeutic higher order structure (Madsen, J. A. et al., Analytical Chemistry, 2016, 88, 2678-2488). These results illustrate that covalent labeling denaturing methods as described herein can be used to differentiate and/or characterize local protein-protein interaction sites.

### Example 4: Differentiating Localized Protein-Protein Interactions of a Model Fc-Fusion Protein Complex

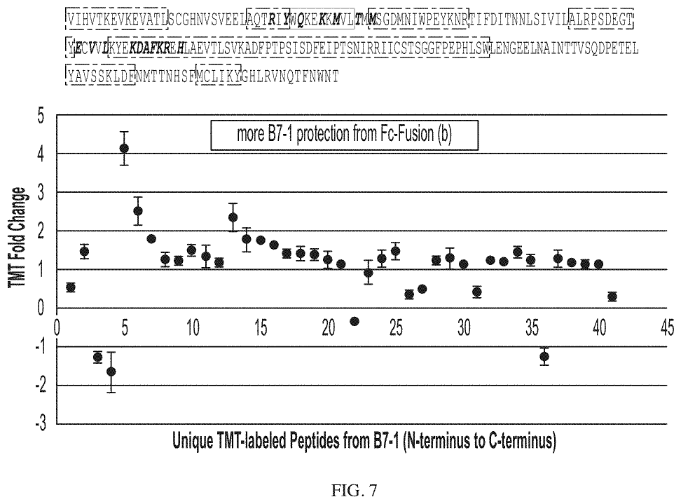

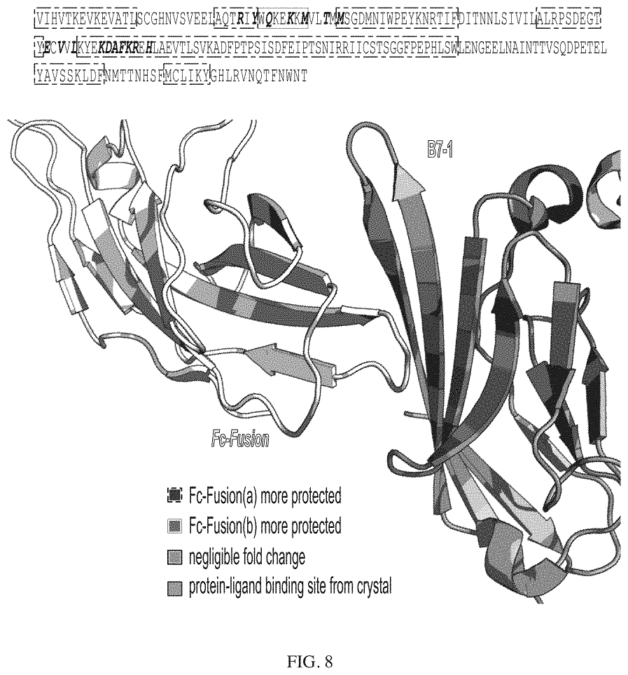

This example describes application of methods of the present disclosure to characterize binding interactions with Fc-Fusion proteins. Two related drugs were utilized for PPI analysis: a model Fc-Fusion(a) and a model Fc-Fusion(b). Both these biologics bind tightly to multiple immune-regulatory ligands, including an exemplary antigen, B7-1 (Stamper, C. C. et al. Nature, 2001, 410, 608-611; Zhang, X. et al., PNAS, 2003, 100, 2586-2591); however, Fc-Fusion(b) has two engineered sequence mutations that yields higher binding affinity as compared to Fc-Fusion(a). B7-1 is decorated with eight known N-linked glycosylation sites, which makes localized PPI differentiation exceedingly challenging. In this experiment, TMT 126, 127, and 128 were reacted with three replicates of B7-1/Fc-Fusion(b) aliquots; and TMT 129, 130, and 131 were reacted with three replicates of B7-1/Fc-Fusion(a) aliquots (the denaturing labeling conditions of 5.3 mM TMT were used for each condition). A Byonic glycopeptide identification feature within our MS data analysis also enabled enhancement of the identification of TMT-labeled glycosylated peptides. The labeled coverage for B7-1 (top) and localized covalent labeling denaturation structural assessment (bottom) for B7-1/Fc-Fusion(a) versus B7-1/Fc-Fusion(b) complexes is shown in FIG. 7. Unlike the previous TNFα complex comparison where all binding sites were covered by at least one TMT-labeled peptide, four of the known protein-ligand contact sites were not covered for the B7-1/Fc-Fusion complexes, a probable consequence of the chymotrypsin specificity, which generated short peptide segments consisting of unmodifiable amino acid residues at these particular B7-1 regions. Regardless, 77% of the known contact sites were covered, and importantly, sizeable differentiation of the two B7-1 complexes was attained. Most of the known protein-protein interaction sites yielded negligible fold changes between B7-1/Fc-Fusion(a) and B7-1/Fc-Fusion(b). However, one specific localized section, WQKEKKMVL (SEQ ID NO: 20), showed a fold change of >4 indicating that B7-1/Fc-Fusion(b) exhibited more label protection, a result consistent with the known higher B7-1 affinity of Fc-Fusion(b) compared to Fc-Fusion(a). This localized section is highlighted in red on the three-dimensional structure of the complex as seen in FIG. 8, and resides in the B7-1 loop region of the protein interaction interface. Thus, this example demonstrates that the provided methods can effectively characterize protein-protein interactions with multiple different type of biotherapeutics.

### Example 5: Differentiating Localized Protein-Protein Interactions Between Stressed and Unstressed Therapeutic Fc-Fusion Proteins

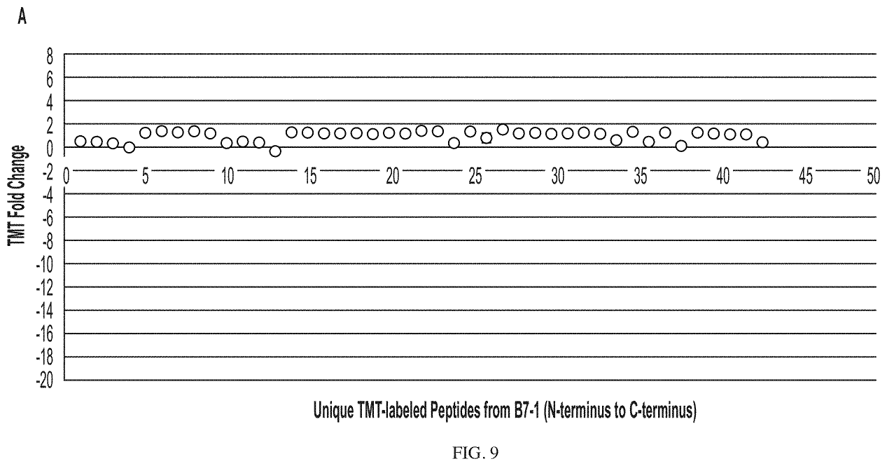

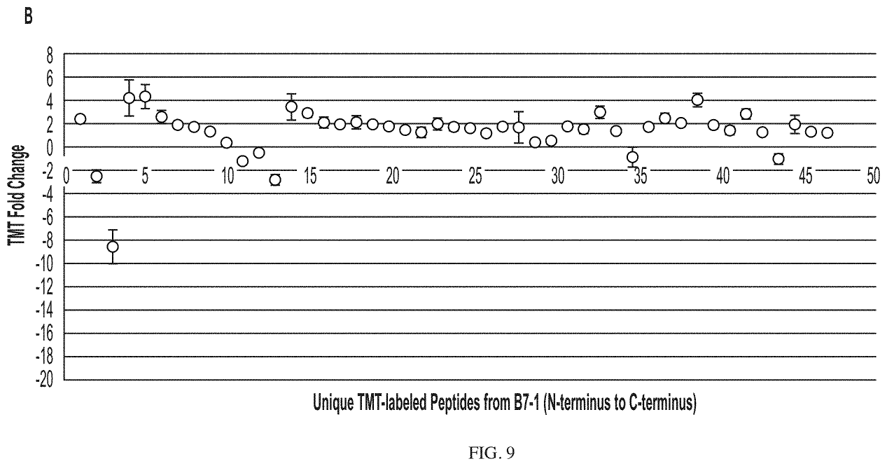

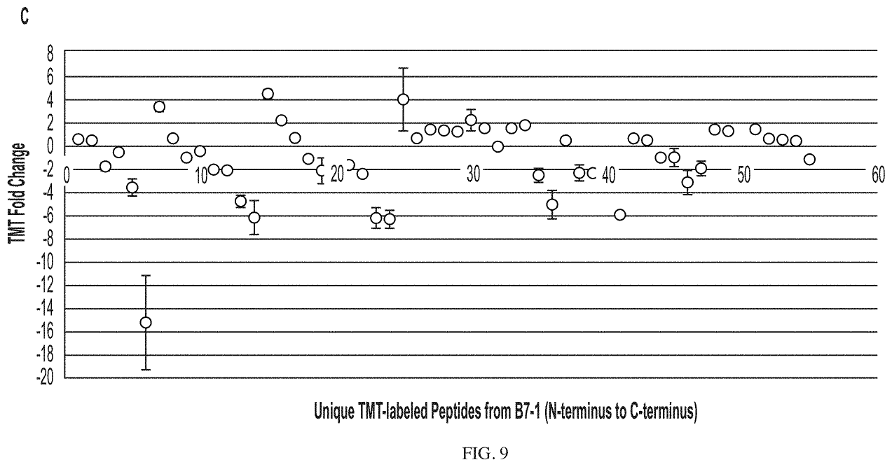

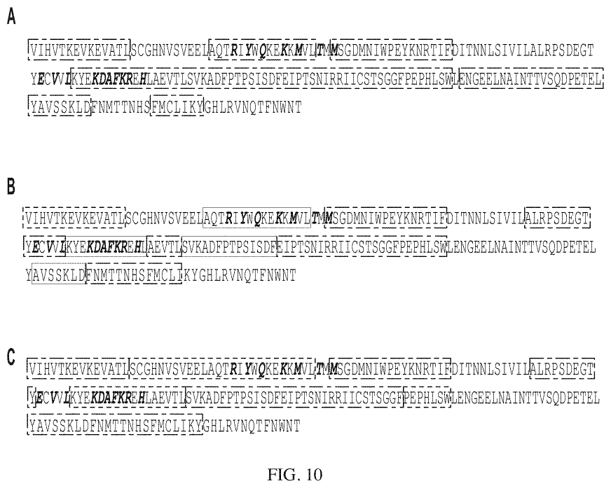

To assess the potential functional implications of unforeseen protein modifications, the binding behavior of an exemplary B7-1/Fc-Fusion(a) complex was further characterized by stressing Fc-Fusion(a) (e.g., exposing to a stressor, such as acidic conditions, oxidizing conditions, and/or high temperature) and comparing its localized binding to unstressed Fc-Fusion(a). Acid (pH 3 for 1.5 hours), oxidizing (0.2% hydrogen peroxide for 1 hour), and heat (55° C. for 18 hours) stressed Fc-Fusion(a) were separately bound to B7-1, and analyzed as follows: TMT 126, 127, and 128 were reacted with three replicates of B7-1/stressed (e.g., acidified, oxidized, or heated) Fc-Fusion(a) aliquots and TMT 129, 130, and 131 were reacted with three replicates of B7-1/Fc-Fusion(a) aliquots. The analysis procedures were performed similarly to the previous section. FIG. 9 panel A illustrates the localized covalent labeling denaturation structural assessment, and FIG. 10 at (A) shows the analogous TMT-labeled peptide sequence coverage map for the acidified sample comparison. As seen in the figures, all B7-1 TMT-labeled peptides had negligible fold changes (i.e., less than two) indicating that the acidified Fc-fusion protein did not affect B7-1 binding. Interestingly, size exclusion chromatography (SEC) of this stressed Fc-Fusion(a) sample showed a substantial increase in aggregate formation (data not shown); however, based on the covalent labeling denaturation data, the aggregated species likely possess a native-like conformation, and behave functionally in a similar manner as the unstressed sample. While native-like aggregates may have similar function to its monomeric counterpart, there is still an increased risk for an immunogenic response. Protein-protein interaction results of the oxidized Fc-Fusion(a) were dramatically different compared to the acidified sample. Under the oxidizing conditions, many of the methionines on Fc-Fusion(a) were almost fully oxidized; however, no significant increase in aggregation formation was observed by SEC (data not shown). The binding interactions of this stressed sample to B7-1 exhibited dramatic differences compared to the B7-1/unstressed Fc-Fusion(a) complex as shown in FIG. 9 panel B and FIG. 10 at (B). Unexpectedly, many of the localized contact areas were shown to be significantly more protected from the label for the complex containing the oxidized fusion protein, and several of these areas were even outside the known protein-ligand interaction locations. That is, the binding of the oxidized therapeutic was likely stronger than its unstressed counterpart in certain B7-1 regions; however, the specificity of the localized interactions have been drastically altered and very likely drug functionality has changed. As a last comparison, heat-stressed Fc-Fusion(a) in complex with B7-1 was assessed. Significant increases in both asparagine deamidation and aggregate formation were observed from PTM and SEC analysis, respectively (data not shown). The results of the covalent labeling denaturation characterization are illustrated in FIG. 9 panel C and FIG. 10 at (C). Overall, the heat-stressed Fc-Fusion(a) complex showed significantly less label protection at the known protein-ligand binding sites as compared to the B7-1/unstressed Fc-Fusion(a) complex. TMT-labelled peptides spanning the amino acid range of WQKEKKMVL (SEQ ID NO: 20) showed a mix of both positive and negative fold changes—peptides with only one label per peptide generally produced the most positive fold changes while peptides with up to three labels per peptide generated the highest negative fold changes. Yet, the magnitude of the fold changes were highest for the negative fold changes, which suggests that the B7-1/unstressed Fc-Fusion(a) complex exhibited more label protection.

In contrast, binding analysis methods such as surface plasmon resonance (SPR) are limited at least in part because they can only assess whole complex interactions (i.e., sum of all the interactions between two proteins). To illustrate this difference, binding affinity for an exemplary B7-1/Fc-Fusion(a) complex, an exemplary B7-1/Fc-Fusion(b) complex and the exemplary B7-1/Fc-Fusion(a) exposed to different stressors was determined by SPR analysis and compared with an exemplary covalent labeling denaturation LC-MS/MS as described herein. For surface plasmon resonance results, K_{D} fold changes were calculated by dividing the appropriate K_{D} values for each set of experiments. Label denaturation results show TMT peptides that passed the following criteria: |fold change|−|error|>2.0. Negative versus positive TMT fold changes indicate that the target protein was more protected from the label (stronger binding) in the complex listed on top versus bottom, respectively, in the far left column for each set of experiments. A summary of the binding affinities as determined by SPR analysis and covalent labeling denaturation LC-MS/MS are shown below in Table 3 and Table 4, respectively, illustrating that the LC-MS/MS method has high sensitivity and that the LC-MS/MS method has enhanced resolution compared to SPR analysis.

TABLE 3
SPR analysis of model B7-1/Fc-Fusion complexes with
and without exposure to different stressors

| | | | | K_{D} Fold |
|---|---|---|---|---|
| Sample | ka (1/Ms) | kd (1/s) | K_{D} (M) | Change |
| B7-1/Fc-Fusion(a) | 4.90E+06 | 1.05E−03 | 2.14E−10 | |
| B7-1/Fc-Fusion(b) | 4.96E+06 | 2.41E−04 | 4.86E−11 | 4.40 |
| B7-1/Fc-Fusion(a) | 3.57E+06 | 1.10E−03 | 3.07E−10 | |
| B7-1/acidified Fc-Fusion(a) | 2.66E+06 | 1.05E−03 | 3.93E−10 | 1.28 |
| B7-1/oxidized Fc-Fusion(a) | 3.15E+06 | 1.95E−03 | 6.20E−10 | 2.02 |
| B7-1/heated Fc-Fusion(a) | 1.91E+06 | 1.20E−03 | 6.26E−10 | 2.04 |

TABLE 4
covalent labeling denaturation LC-MS/MS
analysis of model B7-1/Fc-Fusion complexes
with exposure to different stressors

| | | | # of | TMT |
|---|---|---|---|---|
| | | SEQ ID | TMT | Fold |
| Sample | Peptide | NOS | Labels | Change |
| B7-1/Fc-Fusion(a) | [Y].WQKEKKM.[V] | 21 & 22 | 2 | 4.12 |
| B7-1/Fc-Fusion(b) | [Y].WQKEKKMVL.[T] | 23 & 24 | 2 | 2.49 |
| B7-1/Fc-Fusion(a) | no peptides with | | | |
| | fold changes >2.0 | | | |
| B7-1/acidified Fc- | | | | |
| Fusion(a) | | | | |
| B7-1/Fc-Fusion(a) | [-].VIHVTKEVKEVATL.[S] | 25 & 26 | 1 | 2.42 |
| B7-1/oxidized Fc- | [-].VIHVTKEVKEVATL.[S] | 25 & 26 | 1 | -8.55 |
| Fusion(a) | | | | |
| | [L].AQTRIYW.[Q] | 27 & 28 | 1 | 4.23 |
| | [Y].WQKEKKM.[V] | 21 & 22 | 2 | 4.36 |
| | [Y].WQKEKKMVL.[T] | 23 & 24 | 2 | 2.61 |
| | [Y].WQKEKKMVL.[T] | 23 & 24 | 3 | -2.80 |
| | [W].QKEKKMVL.[T] | 29 & 30 | 1 | 3.47 |
| | [W].QKEKKM.[V] | 31 & 32 | 2 | 2.93 |
| | [L].KYEKDAF.[K] | 33 & 34 | 2 | 3.01 |
| | [F].KREHL.[A] | 35 & 36 | 1 | 2.49 |
| | [L].SVKADFPTPSISDF.[E] | 37 & 38 | 2 | 4.07 |
| | [Y].AVSSKLDF.[N] | 39 & 40 | 1 | 2.87 |
| B7-1/Fc-Fusion(a) | [L].SCGHNVSVEEL.[A] | 41 & 42 | 1 | -3.70 |
| B7-1/heated Fc- | [L].AQTRIYW.[Q] | 27 & 28 | 1 | -15.33 |
| Fusion(a) | | | | |
| | [Y].WQKEKKM.[V] | 21 & 22 | 1 | 3.24 |
| | [Y].WQKEKKMVL.[T] | 23 & 24 | 2 | -2.23 |
| | [Y].WQKEKKMVL.[T] | 23 & 24 | 3 | -4.89 |
| | [Y].WQKEKKM.[V] | 21 & 22 | 3 | -6.29 |
| | [W].QKEKKM.[V] | 31 & 32 | 1 | 4.35 |
| | [W].QKEKKMVL.[T] | 29 & 30 | 2 | -2.55 |
| | [W].QKEKKM.[V] | 31 & 32 | 3 | -6.33 |
| | [W].QKEKKMVL.[T] | 29 & 30 | 3 | -6.44 |
| | [L].KYEKDAF.[K] | 33 & 34 | 2 | -2.66 |
| | [L].KYEKDAF.[K] | 33 & 34 | 3 | -5.18 |
| | [F].KREHLAEVTL.[S] | 43 & 44 | 1 | -2.47 |
| | [F].KREHL.[A] | 35 & 36 | 1 | -2.52 |
| | [F].KREHLAEVTL.[S] | 43 & 44 | 2 | -6.05 |
| | [F].PEPHLSW.[L] | 45 & 46 | 1 | -3.27 |

These results, as well as those previously described herein, highlight the differentiating capability of covalent labeling denaturation methods of the present disclosure across multiple protein-protein interactions of therapeutic relevance.

### Example 6: Manufacture of a Biosimilar Antibody

A batch of a test biologic is produced as a drug substance. A sample of the test biologic is exposed to a binding partner to produce a protein-protein complex in a first state. This complex is exposed to a stressor to obtain a labeled test protein-protein complex in a second state. Mass spectrometry is used to obtain test MS signals of the labeled test protein-protein complex in the first state and the second state, which can be compared to determine interaction sites (i.e., protein-protein binding sites). Further samples of the test biologic in a protein-protein complex can be tested with varying amounts of stressor to assess the strength of one or more local protein-protein interactions. The interaction sites and strength of interactions can be compared with known or determined interactions for a target biologic. If the site and/or strength of interaction between a test biologic and its binding partner are sufficiently similar to that of a target biologic, then the batch of test biologic is processed as a drug product.

### EQUIVALENTS

It is to be understood that while the disclosure has been described in conjunction with the detailed description thereof, the foregoing description is intended to illustrate and not limit the scope of the invention, which is defined by the scope of the appended claims. Other

1. A method of determining a site of protein-protein interaction, comprising:
exposing a first sample of a protein-protein complex with a first level of a label to obtain a labeled protein-protein complex in a first state;
exposing a second sample of the protein-protein complex with a second level of the label to obtain a labeled protein-protein complex in a second state, wherein the second level of the label is sufficient to induce a conformational change of the protein-protein complex;
using mass spectrometry to obtain a MS signal of the labeled protein-protein complex in the first state and a MS signal of the labeled protein-protein complex in the second state; and
determining a site of interaction by comparing the MS signals of the labeled protein-protein complex in the first state and the second state.

2. The method of claim 1, wherein the label is a covalent label.

3. The method of claim 1, wherein the label is an isobaric label.

4. The method of claim 3, wherein the isobaric label is a TMT label.

5. The method of claim 1, wherein using mass spectrometry comprises digesting the labeled protein-protein complex to produce a plurality of labeled peptides.

6. The method of claim 1, wherein the second level of the label is within a range of 100-100,000 molar excess relative to the protein-protein complex.

7. The method of claim 1, wherein the site of interaction is a sequence of the first protein and/or second protein that is protected from labeling (e.g., protected from labeling in the first state, but not in the second state).

8. The method of claim 1, wherein the first and/or second protein is glycosylated.

9. The method of claim 1, further comprising:
exposing the protein-protein complex in the first state to a third level of label to obtain a labeled protein-protein complex in a third state, wherein the third level is sufficient to induce a conformational change of the protein-protein complex;
using mass spectrometry to obtain a MS signal of the labeled protein-protein complex in the third state;
comparing the MS signal of the labeled protein-protein complex in the first, second, and third states to assess binding strength of the first protein to the second protein at one or more sites of interaction.

10. A method of characterizing protein-protein interactions, comprising:
providing a sample of a protein-protein complex comprising a first protein and a second protein;
exposing the protein-protein complex to 2 or more levels of label to obtain labeled protein-protein complexes in 2 or more states, wherein each state corresponds to a level of label, and wherein at least one level of label induces a conformational change of the protein-protein complex;
using mass spectrometry to obtain a MS signal for each of the 2 or more states of labeled protein-protein complex; and
comparing the MS signals to characterize one or more sites of interaction between the first and second protein of the protein complex.

11. The method of claim 10, wherein the label is a covalent label.

12. The method of claim 10, wherein the label is an isobaric label.

13. The method of claim 12, wherein the isobaric label is a TMT label.

14. The method of claim 10, wherein using mass spectrometry comprises digesting the labeled protein-protein complex to produce a plurality of labeled peptides.

15. The method of claim 10, wherein a level of label that induces a conformational change is within a range of 100-100,000 molar excess relative to the protein-protein complex.

16. The method of claim 10, wherein the first and/or second protein is glycosylated.

17. The method of claim 10, wherein protein-protein complex is exposed to 3, 4, 5, 6, 7, 8, 9, 10 or more levels of label.

18. The method of claim 10, wherein characterizing one or more sites of interaction between the first and second protein of the protein complex comprises determining an amino acid sequence of a site of interaction.

19. The method of claim 10, wherein a site of interaction comprises a sequence of the first protein and/or second protein that is protected from labeling in one or more states.

20. The method of claim 10, wherein the method further comprises determining a strength of interaction between the first protein and the second protein at one or more sites of interaction.

21. The method of claim 10, wherein the method comprises:
exposing the protein-protein complex to 3 or more levels of label to obtain labeled protein-protein complexes in 3 or more states, wherein each state corresponds to a level of label, and wherein at least 2 levels of label induce a conformational change of the protein-protein complex;
using mass spectrometry to obtain a MS signal for each of the 3 or more states of labeled protein-protein complex;
comparing the MS signals for each of the 3 or more different states to determine a strength of interaction between the first protein and the second protein at one or more sites of interaction.

22. A method of identifying and/or screening a protein binding partner, comprising
providing a sample of a protein;
contacting the sample of the protein with a test protein to form a protein-test protein complex;
exposing the protein-test protein complex to 2 or more levels of label to obtain labeled protein-protein complexes in 2 or more states, wherein each state corresponds to a level of label, and wherein at least one level of label induces a conformational change of the protein-test protein complex;
using mass spectrometry to obtain a MS signal for each of the 2 or more states of labeled protein-test protein complex; and
determining a site of interaction by comparing the MS signals of the 2 or more states of labeled protein-test protein complex; and
selecting the test protein as a protein binding partner if the site of interaction is tolerable.

23. The method of claim 22, wherein the site of interaction is a sequence of the protein that is protected from labeling.

24. The method of claim 22, wherein the site of interaction is tolerable when it overlaps a desired or predetermined site of interaction between the protein and the protein binding partner.

25. The method of claim 22, wherein the site of interaction is tolerable when the sequence of the protein binding partner that is protected from labeling is 80%, 85%, 90% 95%, 98%, 99% or 100% identical to a desired or predetermined sequence of interaction.

26. The method of claim 22, wherein the label is a covalent label.

27. The method of claim 26, wherein the label is an isobaric label.

28. The method of claim 27, wherein the isobaric label is a TMT label.

29. The method of claim 22, wherein using mass spectrometry comprises digesting the labeled protein-test protein complex to produce a plurality of labeled peptides.

30. The method of claim 22, wherein a level of label sufficient to induce a conformational change is within a range of 100-100,000 molar excess relative to the protein-test protein complex.

31. The method of claim 22, further comprising:
determining a strength of interaction between the protein and the test protein at one or more sites of interaction.

32. The method of claim 22, wherein the protein and/or test protein are glycosylated.


SEQUENCE LISTING (48 sequences)

SEQ ID NO: 1 - protein, 19 aa, Artificial Sequence
  Description of Artificial Sequence Synthetic peptide
SAEINRPDYLDFAESGQVY

SEQ ID NO: 2 - protein, 13 aa, Artificial Sequence
  Description of Artificial Sequence Synthetic peptide
FKGQGCPSTHVLL

SEQ ID NO: 3 - protein, 15 aa, Artificial Sequence
  Description of Artificial Sequence Synthetic peptide
LFKGQGCPSTHVLLT

SEQ ID NO: 4 - protein, 12 aa, Artificial Sequence
  Description of Artificial Sequence Synthetic peptide
FKGQGCPSTHVL

SEQ ID NO: 5 - protein, 14 aa, Artificial Sequence
  Description of Artificial Sequence Synthetic peptide
LFKGQGCPSTHVLL

SEQ ID NO: 6 - protein, 12 aa, Artificial Sequence
  Description of Artificial Sequence Synthetic peptide
KGQGCPSTHVLL

SEQ ID NO: 7 - protein, 14 aa, Artificial Sequence
  Description of Artificial Sequence Synthetic peptide
FKGQGCPSTHVLLT

SEQ ID NO: 8 - protein, 11 aa, Artificial Sequence
  Description of Artificial Sequence Synthetic peptide
KGQGCPSTHVL

SEQ ID NO: 9 - protein, 13 aa, Artificial Sequence
  Description of Artificial Sequence Synthetic peptide
FKGQGCPSTHVLL

SEQ ID NO: 10 - protein, 11 aa, Artificial Sequence
  Description of Artificial Sequence Synthetic peptide
THTISRIAVSY

SEQ ID NO: 11 - protein, 13 aa, Artificial Sequence
  Description of Artificial Sequence Synthetic peptide
LTHTISRIAVSYQ

SEQ ID NO: 12 - protein, 6 aa, Artificial Sequence
  Description of Artificial Sequence Synthetic peptide
QTKVNL

SEQ ID NO: 13 - protein, 8 aa, Artificial Sequence
  Description of Artificial Sequence Synthetic peptide
YQTKVNLL

SEQ ID NO: 14 - protein, 7 aa, Artificial Sequence
  Description of Artificial Sequence Synthetic peptide
QTKVNLL

SEQ ID NO: 15 - protein, 9 aa, Artificial Sequence
  Description of Artificial Sequence Synthetic peptide
YQTKVNLLS

SEQ ID NO: 16 - protein, 12 aa, Artificial Sequence
  Description of Artificial Sequence Synthetic peptide
SAEINRPDYLDF

SEQ ID NO: 17 - protein, 14 aa, Artificial Sequence
  Description of Artificial Sequence Synthetic peptide
LSAEINRPDYLDFA

SEQ ID NO: 18 - protein, 19 aa, Artificial Sequence
  Description of Artificial Sequence Synthetic peptide
SAEINRPDYLDFAESGQVY

SEQ ID NO: 19 - protein, 21 aa, Artificial Sequence
  Description of Artificial Sequence Synthetic peptide
LSAEINRPDYLDFAESGQVYF

SEQ ID NO: 20 - protein, 9 aa, Artificial Sequence
  Description of Artificial Sequence Synthetic peptide
WQKEKKMVL

SEQ ID NO: 21 - protein, 7 aa, Artificial Sequence
  Description of Artificial Sequence Synthetic peptide
WQKEKKM

SEQ ID NO: 22 - protein, 9 aa, Artificial Sequence
  Description of Artificial Sequence Synthetic peptide
YWQKEKKMV

SEQ ID NO: 23 - protein, 9 aa, Artificial Sequence
  Description of Artificial Sequence Synthetic peptide
WQKEKKMVL

SEQ ID NO: 24 - protein, 11 aa, Artificial Sequence
  Description of Artificial Sequence Synthetic peptide
YWQKEKKMVLT

SEQ ID NO: 25 - protein, 14 aa, Artificial Sequence
  Description of Artificial Sequence Synthetic peptide
VIHVTKEVKEVATL

SEQ ID NO: 26 - protein, 15 aa, Artificial Sequence
  Description of Artificial Sequence Synthetic peptide
VIHVTKEVKEVATLS

SEQ ID NO: 27 - protein, 7 aa, Artificial Sequence
  Description of Artificial Sequence Synthetic peptide
AQTRIYW

SEQ ID NO: 28 - protein, 9 aa, Artificial Sequence
  Description of Artificial Sequence Synthetic peptide
LAQTRIYWQ

SEQ ID NO: 29 - protein, 8 aa, Artificial Sequence
  Description of Artificial Sequence Synthetic peptide
QKEKKMVL

SEQ ID NO: 30 - protein, 10 aa, Artificial Sequence
  Description of Artificial Sequence Synthetic peptide
WQKEKKMVLT

SEQ ID NO: 31 - protein, 6 aa, Artificial Sequence
  Description of Artificial Sequence Synthetic peptide
QKEKKM

SEQ ID NO: 32 - protein, 8 aa, Artificial Sequence
  Description of Artificial Sequence Synthetic peptide
WQKEKKMV

SEQ ID NO: 33 - protein, 7 aa, Artificial Sequence
  Description of Artificial Sequence Synthetic peptide
KYEKDAF

SEQ ID NO: 34 - protein, 9 aa, Artificial Sequence
  Description of Artificial Sequence Synthetic peptide
LKYEKDAFK

SEQ ID NO: 35 - protein, 5 aa, Artificial Sequence
  Description of Artificial Sequence Synthetic peptide
KREHL

SEQ ID NO: 36 - protein, 7 aa, Artificial Sequence
  Description of Artificial Sequence Synthetic peptide
FKREHLA

SEQ ID NO: 37 - protein, 14 aa, Artificial Sequence
  Description of Artificial Sequence Synthetic peptide
SVKADFPTPSISDF

SEQ ID NO: 38 - protein, 16 aa, Artificial Sequence
  Description of Artificial Sequence Synthetic peptide
LSVKADFPTPSISDFE

SEQ ID NO: 39 - protein, 8 aa, Artificial Sequence
  Description of Artificial Sequence Synthetic peptide
AVSSKLDF

SEQ ID NO: 40 - protein, 10 aa, Artificial Sequence
  Description of Artificial Sequence Synthetic peptide
YAVSSKLDFN

SEQ ID NO: 41 - protein, 11 aa, Artificial Sequence
  Description of Artificial Sequence Synthetic peptide
SCGHNVSVEEL

SEQ ID NO: 42 - protein, 13 aa, Artificial Sequence
  Description of Artificial Sequence Synthetic peptide
LSCGHNVSVEELA

SEQ ID NO: 43 - protein, 10 aa, Artificial Sequence
  Description of Artificial Sequence Synthetic peptide
KREHLAEVTL

SEQ ID NO: 44 - protein, 12 aa, Artificial Sequence
  Description of Artificial Sequence Synthetic peptide
FKREHLAEVTLS

SEQ ID NO: 45 - protein, 7 aa, Artificial Sequence
  Description of Artificial Sequence Synthetic peptide
PEPHLSW

SEQ ID NO: 46 - protein, 9 aa, Artificial Sequence
  Description of Artificial Sequence Synthetic peptide
FPEPHLSWL

SEQ ID NO: 47 - protein, 157 aa, Artificial Sequence [sequence omitted]

SEQ ID NO: 48 - protein, 199 aa, Artificial Sequence
  Description of Artificial Sequence Synthetic polypeptide
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNT